(19) United States
(12) Patent Application Publication
(10) Pub. No.: US 2025/0002546 A1
(43) Pub. Date: Jan. 2, 2025

(54) Vectors for Increasing NPRL2 Expression in Cancer Cells and Methods of Use Thereof

(71) Applicant: Genprex, Inc., Austin, TX (US)

(72) Inventor: Michael Redman, Austin, TX (US)

(21) Appl. No.: 18/710,542

(22) PCT Filed: Nov. 16, 2022
(86) PCT No.: PCT/US22/79978

§ 371 Date: May 15, 2024

Related U.S. Application Data
(60) Provisional application No. 63/279,871, filed on Nov. 16, 2021.

(51) Int. Cl. C07K 14/47; A61K 38/17; A61K 48/00; A61P 35/00; C12N 15/85

(52) U.S. Cl. CPC C07K 14/4703; A61K 38/1709; A61K 48/005; A61P 35/00; C12N 15/85; C12N 2830/42; C12N 2830/50

(57) ABSTRACT

The disclosure provides nucleic acid constructs including a polynucleotide sequence encoding a NPRL2 protein. Also contemplated are nonviral and viral vectors including the nucleic acid constructs. The nonviral vectors include. e.g., DOTAP: cholesterol liposomes. Further contemplated herein are compositions including the vectors and methods of using the compositions for the treatment of cancer in a human subject in need thereof. These methods can further include administering a second anti-cancer therapy to the subjects in need thereof.

### CROSS-REFERENCE TO RELATED APPLICATIONS

This application claims the benefit of U.S. Provisional Application No. 63/279,871, filed Nov. 16, 2021, which is incorporated herein by reference in its entirety.

### SEQUENCE LISTING

This application includes a Sequence Listing submitted electronically as an xml file named 198628-46176-Seq-Listing.xml, created on Nov. 16, 2022, with a size of 14.5 kilobytes. The Sequence Listing is incorporated herein by reference.

### FIELD OF THE INVENTION

The present disclosure relates to vectors that include a nucleotide sequence encoding a NPRL2 protein. Also contemplated are compositions including nonviral and viral vectors for increasing NPRL2 expression in cancer cells. Further contemplated are methods of treating cancer in a human subject including administration of the nonviral or viral vectors and compositions to the subject.

### BACKGROUND

The nitrogen permease regulator-like 2 gene (NPRL2), also known as tumor suppressor candidate 4 (TUSC4) encodes a protein that is associated with the Gap activity toward rags 1 (GATOR1) complex. The NPRL2 protein regulates pathways including the mTOR signaling pathway and the PI3K/Akt signaling pathway. NPRL2 also acts as a tumor suppressor by regulating signaling pathways and other processes that are frequently associated with cancer progression. For example, the PDK1 signaling pathway is involved in promoting cell proliferation, and cancer cells frequently develop mechanisms to increase proliferation by increasing PDK1 signaling. NPRL2 plays a tumor inhibiting role by inhibiting PDK1 signaling. As another example, the Breast Cancer Gene (BRCA1) encodes a protein that functions to maintain genomic stability by regulating pathways responsible for detecting and repairing damaged DNA. NPRL2 can also inhibit cancer progression by enhancing stabilization of the protein encoded by the BRCA1 gene. Since NPRL2 has tumor suppressing activities, its expression is often down regulated in cancer cells.

Thus, development of an effective therapy for activating or restoring NPRL2 in cancer cells by increasing NPRL2 expression and/or overcoming NPRL2 inhibition that is less toxic than available therapies would provide a great advance.

### SUMMARY OF THE DISCLOSURE

In one aspect, the disclosure provides a codon optimized polynucleotide sequence encoding a human NPRL2 protein, wherein the polynucleotide sequence comprises SEQ ID NO: 1.

In embodiments, the disclosure provides a nucleic acid construct comprising a polynucleotide sequence encoding a human NPRL2 protein, and further comprising a CMV promoter operably linked to the polynucleotide sequence encoding the human NPRL2 protein. In embodiments the nucleic acid construct includes a CMV promoter comprising a sequence having greater than 90% sequence identity to SEQ ID NO: 2. In embodiments, the nucleic acid construct includes a CMV promoter that comprises SEQ ID NO: 2.

In embodiments, the nucleic acid construct further comprises a CMV enhancer. In embodiments the CMV enhancer comprises a sequence having greater than 90% sequence identity to SEQ ID NO: 3. In embodiments, the CMV enhancer comprises SEQ ID NO: 3.

In embodiments, the nucleic acid construct further comprises a Human T-cell leukemia virus type I (HTLV-I) regulatory sequence. In embodiments, the HTLV-I regulatory sequence comprises a sequence having greater than 90% sequence identity to SEQ ID NO: 4. In embodiments, the HTLV-I regulatory sequence comprises SEQ ID NO: 4.

In embodiments, the nucleic acid construct further comprises a bovine growth hormone polyadenylation (BGH polyA) sequence. In embodiments, the BGH polyA sequence comprises a sequence having greater than 90% sequence identity to SEQ ID NO: 5. In embodiments, the BGH polyA sequence comprises SEQ ID NO: 5.

In embodiments, the nucleic acid construct further comprises at least one intron. In embodiments, the at least one intron is β-globin intron. In embodiments, intron sequence comprises a sequence having greater than 90% sequence identity to SEQ ID NO: 6. In embodiments, the β-globin intron sequence comprises SEQ ID NO: 6. In embodiments, the nucleic acid construct further comprises one or more splicing enhancer sequences.

In embodiments, the nucleic acid construct further comprises a bacterial backbone sequence. In embodiments, the bacterial backbone sequence comprises a sequence having greater than 90% sequence identity to SEQ ID NO: 7. In embodiments, the bacterial backbone sequence comprises SEQ ID NO: 7. In embodiments, the bacterial backbone sequence comprises a R6K origin sequence. In embodiments, the bacterial backbone sequence comprises a selectable marker sequence. In embodiments, the selectable marker sequence is not an antibiotic resistance marker, and may be, e.g., an RNA-OUT sequence. In embodiments, the selectable marker is an antibiotic resistance marker.

In embodiments, the nucleic acid construct comprises a sequence having greater than 90% sequence identity to SEQ ID NO: 8. In embodiments, the nucleic acid construct comprises SEQ ID NO: 8.

In embodiments, the nucleic acid construct comprises a nucleic acid sequence encoding a NPRL2 protein comprising a sequence that is at least 90% identical to SEQ ID NO: 9. In embodiments, the nucleic acid construct comprises a nucleic acid sequence encoding a NPRL2 protein comprising SEQ ID NO: 9.

In another aspect, the disclosure provides a nonviral vector including a nucleic acid construct encoding a NPRL2 protein as described herein. In embodiments the nonviral vector is a liposomal nonviral vector. Such liposomes can include 1,2-dioleoyl-3-trimethylammonium-propane (DOTAP) and cholesterol. The DOTAP: cholesterol molar ratio can be between about 3:1 and about 1:3. In embodiments, the disclosure provides a nonviral vector comprising the nucleic acid construct and a DOTAP: cholesterol liposome. In embodiments of the nonviral vector, the DOTAP: cholesterol molar ratio is between about 3:1 and about 1:3 and the nucleic acid construct comprises SEQ ID NO: 1. In embodiments of the nonviral vector, the DOTAP: cholesterol molar ratio is between about 3:1 and about 1:3 and the nucleic acid construct comprises SEQ ID NO: 8.

In embodiments, the disclosure provides a pharmaceutical composition comprising the nonviral vector as described herein and a pharmaceutically acceptable excipient. In embodiments, the pharmaceutical composition further comprises about 5% dextrose, about 0.9% sodium chloride or a combination of both. In embodiments, the DOTAP: cholesterol liposome has a particle size range of about 40 nanometers to about 6 micrometers. In embodiments, the average DOTAP: cholestorol liposome has a particle size of about 145 nanometers. In embodiments, the average DOTAP: cholesterol liposome has a particle size of 60 to 120 nanometers after microfluidization.

In embodiments, the disclosure provides a method for treating cancer in a human subject comprising administering to the human subject in need thereof a pharmaceutical composition comprising a nucleic acid construct disclosed herein that expresses NPRL2 (e.g., from the codon optimized NPRL2 coding sequence of claim 1). In embodiments, the method for treating cancer in a human subject comprises administering to the human subject in need thereof a pharmaceutical composition comprising the nonviral vector disclosed herein (e.g., DOTAP/cholesterol liposomes with a NPRL2 expression construct, such as SEQ ID NO: 8). In embodiments, the method for treating cancer in a human subject comprises administering to the human subject in need thereof the pharmaceutical composition disclosed herein. In embodiments, the cancer is selected from the group consisting of: prostate cancer, colon cancer, pancreatic cancer, breast cancer, melanoma, osteosarcoma, neuroblastoma, leukemia, lung cancer, renal cancer and rectal cancer. In embodiments, the cancer is lung cancer, such as small cell or non-small cell lung cancer.

In embodiments, the disclosure provides a method for generating or augmenting an anti-tumor immune response in a human subject, comprising administering to the human subject in need thereof a pharmaceutical composition comprising a nucleic acid construct disclosed herein that expresses NPRL2 (e.g., from the codon optimized NPRL2 coding sequence of claim 1). In embodiments, the method for generating or augmenting an anti-tumor therapeutic immune response comprises administering to the human subject in need thereof a pharmaceutical composition comprising the nonviral vector disclosed herein (e.g., DOTAP/cholesterol liposomes with a NPRL2 expression construct, such as SEQ ID NO: 8). In embodiments, the method for generating or augmenting an anti-tumor immune response comprises administering to the human subject in need thereof the pharmaceutical compositions disclosed herein.

In embodiments, the pharmaceutical composition is administered intravenously or intranasally.

In embodiments, the method further comprises administering a second anti-cancer therapy to the subject. In embodiments, the second anti-cancer therapy comprises at least one of: chemotherapy, radiation treatment, and surgery. In embodiments, the second anti-cancer therapy is a checkpoint inhibitor or a BRAF inhibitor. In embodiments, the second anti-cancer therapy is an EGFR inhibitor. In embodiments, the checkpoint inhibitor is an anti-PD1 antibody or an anti-PDL1 antibody. In embodiments, the checkpoint inhibitor is pembrolizumab and the BRAF inhibitor is encorafenib. In embodiments, the EGFR inhibitor is cetuximab or nivolumab.

In embodiments, the disclosure provides a viral vector comprising a nucleic acid construct encoding NPRL2. In embodiments, the viral vector comprises the NPRL2 encoding sequence of SEQ ID NO: 1. In embodiments, the method for treating cancer in a human subject comprises administering to the human subject in need thereof the viral vector. In embodiments, the viral vector is an Adeno-Associated Virus (AAV) viral vector.

In one aspect, the disclosure provides a nucleic acid construct including (i) a polynucleotide sequence encoding a NPRL2 protein (e.g., SEQ ID NO: 1) flanked by a 5′ untranslated region (UTR) and a 3′ UTR; (ii) a promoter (e.g., a CMV promoter) operably linked to the polynucleotide sequence encoding the NPRL2 protein; and (iii) a selectable marker. In embodiments, the NPRL2 protein comprises the amino acid sequence of SEQ ID NO: 9, or an amino acid sequence having greater than 95% sequence identity to SEQ ID NO: 9. In embodiments, the polynucleotide sequence encoding the NPRL2 protein is a codon optimized sequence. In embodiments, the polynucleotide sequence encodes an amino acid sequence including amino acids 26 to 335 of SEQ ID NO: 9, or an amino acid sequence having greater than 95% sequence identity to amino acids 26 to 335 of SEQ ID NO:9. In some nucleic acid constructs, the selectable marker is a RNA selectable marker (e.g., RNA-OUT, RNAI, a suppressor tRNA). In the nucleic acid constructs, the 5′ UTR can include, for example, at least one intron and/or a HTLV-IR element.

In another aspect, the disclosure provides a viral vector including a nucleic acid construct encoding a NPRL2 protein as described herein. In embodiments, the viral vector is, for example, an adeno-associated virus (AAV) vector, a retroviral vector, a lentiviral vector, an adenoviral vector, etc.

### BRIEF DESCRIPTION OF THE FIGURES

FIGS. 1A, 1B, and 1C. Antitumor effect of NPRL2-DOTAP, anti-PD1 antibody and combination of both on LLC2-luc tumors in syngeneic mice. FIG. 1A provides a diagram summarizing the experimental protocol for obtaining the data presented in FIGS. 1B-1C an in FIGS. 2A, 2B, 2C, 2D, 2E, and 2F. NPRL2-DOTAP refers to an NPRL2 expression construct loaded in DOTAP/cholestorol liposomes. FIG. 1B and FIG. 1C provide tumor volume over time following treatment with NPRL2-DOTAP, anti-PD1 antibody and combination of both.

FIGS. 2A, 2B, 2C, 2D, 2E, and 2F. Immune cell analysis of NPRL2-DOTAP, anti-PD1 antibody and combination of both on LLC2-luc tumors in syngeneic mice (from FIG. 1A). Immune cell analysis from the tumor microenvironment (TME) assayed using FACS. FIG. 2A provides the percent of CD49b positive immune cells in the TME. FIG. 2B provides the percent CD3, CD4, and CD8 positive T cells in the TME. FIG. 2C provides the percent CD3, CD4, and CD8 positive T cells expressing PD1. FIG. 2D provides the percent of Lin negative myeloid cells in the TME. FIG. 2E provides the percent Lin negative, MHCII positive dendritic cells in the TME. FIG. 2F provides the percent CD11b positive/CD11c positive dendritic cells in the TME.

FIGS. 3A, 3B, 3C and 3D. Antitumor effect and immune cell analysis of NPRL2-DOTAP, anti-PD1 antibody and combination of both on H1299-luc tumors in humanized mice. FIG. 3A provides a diagram summarizing the experimental protocol. FIG. 3B provides tumor volume over time following treatment with NPRL2-DOTAP, anti-PD1 antibody and combination of both. FIGS. 3C and 3D provide percent HLA-DR positive dendritic cells and percent CD56+NK cells, respectively, in the TME as measured by FACS following treatment with NPRL2-DOTAP, anti-PD1 antibody and combination of both (order of bars from left to right: control (empty vector), NPRL2-DOTAP, anti-PD1 antibody, combination).

FIGS. 4A, 4B, 4C, and 4D. Effect of NPRL2-DOTAP, anti-PD1 antibody and combination of both on A549 lung metastasis in humanized mice. FIG. 4A provides a diagram summarizing the experimental protocol. FIGS. 4B and 4C provide tumor metastasis as measured by in vivo imaging system (IVIS) at week 4, following treatment with NPRL2-DOTAP, anti-PD1 antibody and combination of both. FIG. 4D provides the IVIS images showing metastasis following control (empty vector) or treatment with NPRL2-DOTAP, anti-PD1 antibody and combination of both.

FIGS. 5A, 5B, 5C, 5D, 5E, 5F, 5G, and 5H. Tumor microenvironment immune analysis on A549 lung metastases in humanized mice following treatment with NPRL2-DOTAP, anti-PD1 antibody and combination of both. Immune cell analysis from the tumor microenvironment (TME) of humanized mice (from FIG. 4A) was assayed using FACS. FIGS. 5A, 5B and 5C provide the percent CD45 cells, the percent CD3 T cells, and the percent CD8 T cells in the TME following treatment with NPRL2-DOTAP, anti-PD1 antibody and combination of both. FIGS. 5D and 5E provide the percent NK cells (CD56 positive) and percent Treg cells in the TME following treatment with NPRL2-DOTAP, anti-PD1 antibody and combination of both. FIGS. 5F and 5G provide the percent of PD1 positive CD8 T cells and the percent of CD69 positive CD8 T cells in the TME following treatment with NPRL2-DOTAP, anti-PD1 antibody and combination of both. FIG. 5H provides the percent of effector memory (EM) (left panel) and central memory (CM) (right panel) CD8 T cells in the TME following treatment with NPRL2-DOTAP, anti-PD1 antibody and combination of both.

### DETAILED DESCRIPTION OF THE DISCLOSURE

Provided herein are nucleic acid constructs including a polynucleotide sequence encoding human NPRL2. Also contemplated are nonviral vectors comprising the nucleic acid constructs disclosed herein and DOTAP: cholesterol liposomes. Additionally contemplated are viral vectors having the polynucleotide sequence encoding human NPRL2 and/or nucleic acid constructs disclosed herein. Further contemplated herein are compositions comprising the nonviral vectors or viral vectors disclosed herein. Also provided herein are methods of using the compositions disclosed herein for the treatment of cancer in a human subject in need thereof. These methods can further include administering a second anti-cancer therapy to the subjects in need thereof, i.e., combination therapy.

The nonviral vectors, viral vectors, and compositions described herein are useful for increasing or restoring NPRL2 expression to NPRL2-deficient cancer cells, thus overcoming down-regulation or loss of NPRL2 related signaling in human tumors. When cancer cells take up the vectors described herein, NPRL2 expression is increased. By increasing NPRL2 in NPRL2-deficient cancer cells, NPRL2 inhibits PDK1 signaling, resulting in decreased proliferation of the cancer cells. Additionally, increasing NPRL2 expression in tumor cells stabilizes BRCA1, increasing genetic stability of cancer cells.

### I. Nucleic Acid Constructs

Polynucleotide sequences described herein include a codon optimized sequence encoding a NPRL2 protein (e.g., SEQ ID NO: 1). In embodiments, nucleic acid constructs described herein include (i) a codon optimized polynucleotide sequence encoding a NPRL2 protein flanked by a 5′ untranslated region (UTR) and a 3′ UTR, and (ii) a promoter (e.g., CMV) operably linked to the polynucleotide sequence encoding the NPRL2 protein. In embodiments, the nucleic acid construct includes a CMV enhancer. In embodiments, the nucleic acid construct includes a HTLV-1 regulatory sequence. In embodiments, the nucleic acid construct further comprises a bovine growth hormone polyadenylation (BGH polyA) sequence in the 3′ UTR. In embodiments, the nucleic acid construct further comprises at least one intron. In embodiments, the nucleic acid construct further comprises one or more splicing enhancer sequences. In embodiments, the nucleic acid construct includes a bacterial plasmid backbone. In embodiments, the nucleic acid construct includes a selectable marker.

In a typical embodiment, the nucleic acid construct is used for recombinant production of human NPRL2 in a cancer cell (e.g., in a subject's cancer cells). Nucleic acid constructs include expression constructs including plasmids. The term “expression construct” refers to a recombinant polynucleotide construct that includes a nucleic acid coding for an RNA capable of being transcribed in a cell. Methods for constructing expression constructs and plasmids through standard recombinant techniques are known in the art. Methods for designing expression constructs/plasmids for gene therapy applications, including antibiotic-free vector production, are also known. Various sequences and elements have been reported to increase and sustain therapeutic protein production (e.g., introns, Kozak consensus). Such sequences and elements are disclosed below under Control/Regulatory Sequences. Expression constructs constructs/plasmids for inclusion in the nonviral vectors described herein can be produced in a suitable host producer cells (e.g., E. coli) using suitable methods, e.g., fed-batch fermentation, batch fermentation, etc. For example, the HyperGRO™ inducible fed-batch fermentation process may be used to manufacture plasmid DNA. The HyperGRO™ process yields plasmid productivity of up to 2,600 mg/L with low levels of nicking or multimerization. High yield of plasmid per gram of bacteria improves final product purity since plasmid is enriched relative to host cell impurities. Boehringer Ingelheim (Vienna, Austria) has developed an alternative high yield fermentation process which is commercially available for cGMP production of plasmid DNA-based vectors. Plasmid DNA can be extracted from fermentation cells using alkaline lysis. Following plasmid production, the plasmid can be purified by processes, such as anion exchange chromatography followed by hydrophobic interaction chromatography, that isolate plasmid DNA away from impurities (e.g., endotoxin, bacterial RNA, genomic DNA).

In some embodiments, a nucleic acid construct as disclosed herein is a plasmid, which contains in addition to the NPRL2 coding sequence, one or more of the the following sequences: RNA-OUT, CMV enhancer/promoter, CMV-human T-lymphotropic virus type I (HLTV-I) R Region Exon 1, HTLV-I R element, β globin intron, splicing enhancer, Kozak sequence, BGH polyA signal, trpA terminator, and origin. The plasmid can also include a bacterial plasmid backbone for production of the plasmid in bacterial cells (in some tissues, bacterial regions of approximately 1,000 bp or more promote transgene silencing). In embodiments, the plasmid is derived from a NTC9385R plasmid (J. A. Williams, Vaccines 2013 1:225-249; Borggren et al., Hum Vaccin Immunother. 2015 11 (8): 1983-1990), which is commercially available (Nature Technologies Corporation, Lincoln, NE, US). In embodiments, a β-globin intron is included for its efficient splice acceptor, and in further embodiments, the splice donor is derived from the upstream HTLV-IR. However, any strong splice acceptor and splice donor could be used. In embodiments, HTLV-I R is included as a translational enhancer. However, any suitable translational enhancer can be used. In embodiments, a splicing enhancer is included within the intron and/or a flanking exon to increase transgene expression through increased intron splicing.

i. NPRL2 Polynucleotide and Amino Acid Sequences

The nucleic acid constructs described herein include a codon optimized polynucleotide sequence of SEQ ID NO: 1. The codon optimized polynucleotide sequence encodes a NPRL2 protein according to SEQ ID NO: 9. In embodiments, the polynucleotide sequence encodes an amino acid sequence including amino acids 26 to 335 of SEQ ID NO: 9, or an amino acid sequence having greater than 95% sequence identity to amino acids 26 to 335 of SEQ ID NO:9.

In embodiments, the polynucleotide sequence encoding human NPRL2 comprises SEQ ID NO: 1, or comprises a polynucleotide sequence having greater than 85%, greater than 90%, greater than 95%, greater than 96%, greater than 97%, greater than 98%, or greater than 99% sequence identity to SEQ ID NO: 1. In embodiments, the NPRL2 protein encoded by the polynucleotide sequence includes the amino acid sequence of SEQ ID NO:9, or an amino acid sequence having greater than 90%, greater than 95%, greater than 96%, greater than 97%, greater than 98%, or greater than 99% sequence identity to SEQ ID NO:9. As used herein, the term “sequence identity” refers to the degree of which two sequences (e.g., peptide, polypeptide, nucleic acid, etc.) have the same sequential composition of monomer subunits.

ii. Control/Regulatory Sequences

The nucleic acid constructs disclosed herein include control and regulatory sequences that are operably linked to the polynucleotide sequence encoding a NPRL2 protein. The nucleic acid constructs disclosed herein can include appropriate control sequences for expression of the human NPRL2 in human cancer cells. “Control sequences” include nucleic acid sequences necessary for replication of a vector in a producer cell (e.g., E. coli cell), as well as nucleic acid sequences necessary for, or involved in, transcription and/or translation of an operably linked NPRL2 coding sequence in a target cell (e.g., a human cancer cell). As used herein, the term “operably linked” refers to a physical or functional juxtaposition of the components so described as to permit them to function in their intended manner. In the example of an expression control element in operable linkage with a polynucleotide sequence encoding a NPRL2 protein, the relationship is such that the control element modulates expression of the NPRL2 protein encoding sequence. Examples of control/regulatory sequences include promoters, enhancers, translation initiation signals, termination signals, polyadenylation sequences (e.g., polyA signals derived from bovine growth hormone, SV40, rabbit β-globin), origins of replication (e.g., the high copy number pUC replication origin which can be reduced to 700 bp without loss of high copy number replication, a non-pUC mini-origin R6K Nanoplasmid™ from Nature Technology Corporation, Lincoln, NE, etc.), Kozak sequences (e.g., GCCACCATG), posttranslational regulatory elements, introns, nuclear targeting sequences, etc. A suitable promoter may be used in the nucleic acid constructs described herein. In embodiments, the CMV promoter/enhancer is used, and serves a dual role as a promoter and an enhancer. In other embodiments, chimeric promoters that are a fusion of two different promoter sequences or a fusion of a promoter sequence and an inducible element can be used. For example, a chicken β-actin/CMV enhancer combination can be used. Promoters, in addition to the CMV promoter, that can be used to promote transcription of the NPRL2 transgene include simian virus 40 (SV40) early promoter, elongation factor-1α, (EF1α), phosphoglycerate kinase (PGK), and human β-actin promoter (ACTB). In some embodiments, a tissue-specific promoter can be used. In some embodiments, a nucleic acid construct as described herein includes one or more (e.g., 1, 2, 3, 4, 5, etc.) introns. For example, in a nucleic acid construct as disclosed herein, the 5′ UTR, 3′ UTR, and/or the NPRL2 coding sequence can include an intron. As another example, a chimeric intron (e.g., from the β-globulin and/or immunoglobulin heavy chain genes) upstream of the coding sequence can be used. Additionally or alternatively, the 5′ UTR can include a HTLV-I R element for enhancement of mRNA translation efficiency and increasing transgene expression. Nuclear targeting sequences, which promote shuttling of the nucleic acid construct into the nucleus, can be included the nucleic acid construct as described herein. MicroRNA target sites that mediate transgene expression in specific tissues or cell lineages and S/MAR regions that promote replication and long-term episomal transgene expression can also be included in some embodiments of a nucleic acid construct as described herein.

iii. Selectable Markers

In embodiments, the nucleic acid constructs as disclosed herein include a selectable marker. A “selectable marker” as used herein is a nucleic acid sequence that confers a trait suitable for selection for a cell containing the nucleic acid construct. Selectable markers can include RNA selectable markers such as RNA-OUT (Luke et al., Vaccine 2009 vol. 27 (46): 6454-6459; Luke et al. Methods Mol Biol. 2014 vol. 1143:91-111), RNAI (U.S. Pat. No. 9,297,014), and suppressor tRNAs (Soubrier et al., Gene Therapy 1999 vol. 6:1482-1488). RNA selectable markers are useful in applications where use of antibiotic-resistance markers is undesirable, including in production of nonviral vectors. For example, some regulatory agencies recommend avoiding inclusion of antibiotic resistance markers in DNA therapies administered to humans due to risk of unintended immune response and transmission of the antibiotic-resistant genes to the patient's enteric bacteria. Thus, in some embodiments of a nucleic acid construct, the selectable marker is not an antibiotic resistance gene. In other embodiments, selectable markers can include an antibiotic resistance gene, for example, genes encoding resistance to ampicillin, chloramphenicol, tetracycline or kanamycin.

### II. Nonviral Vectors

The term “vector” as used herein refers to a vehicle for delivering genetic material (e.g., RNA or DNA) to a cell, including for example, viral vectors (such as AAV and lentiviral vectors) and nonviral vectors. The term “nonviral vector” is used herein to refer to a nonviral vehicle for delivering genetic material to a cell. In embodiments, the nonviral vector comprises one or more carrier molecules (e.g., DOTAP: cholesterol liposome) complexed with a nucleic acid construct (e.g., a plasmid) as disclosed herein. The liposome formulations described herein deliver the nucleic acid construct into the target cell; entering target cells via endocytosis pathways to avoid lysosomal degradation. Once a liposome particle binds to a negatively-charged cancer cell, the nucleic acid construct is transfected into the cell (e.g., via endocytosis) and NPRL2 is expressed. In embodiments, the non-viral vectors described herein result in a high level of transfection efficiency with a low level of toxicity. The nonviral vectors display specificity and protect against degradation of the nucleic acid construct by the target cell during transfection. The liposome formulations are designed for stability, increased half-life of the polynucleotide construct and the prevention of aggregation of the lipid particles. In the liposomal nonviral vectors, the nucleic acid constructs can be added to liposomes in a range of concentrations. The ratio of the nucleic acid construct to lipids (liposomes) can be optimized for transfection efficiency. In embodiments, nucleic acid constructs are added to the liposomes at a concentration of 20, 25, 50, 75, 100, 110, 120, 130, 140, 150, 160, 170, 180, 190, 200, 225, 275, 300, 350, 375, 400, 450, 500, 550, 600, 650, 700, 750, 800, 850, 900, 950, or 1000 μg per 50, 100, 150, 200, 300, 400, 500, 600, 700, 800, 900, 1000, 1100, 1200, 1300, 1400, 1500, 2000, 2500, 3000, 3500, 4000, 4500, 5000, 5500, 6000, 7000, 8000, 9000, or 10,000 μl, as well as 15, 20, 25, 50 ml final volume. These concentrations may vary depending upon the ratio of the liposome components (e.g., DOTAP to cholesterol, cholesterol derivative or cholesterol mixture) in the particular liposome preparation. In some embodiments, equal volumes of nucleic acid construct and lipids (e.g., DOTAP: cholesterol liposome), at a concentration to obtain about 25 μg, 50 μg, 75 μg, 100 μg, 110 μg, 120 μg, 125 μg, 130 μg, 140 μg, 150 μg, 160 μg, 170 μg, 180 μg, 190 μg, 200 μg, 210 μg, 220 μg, 225 μg, 230 μg, 240 μg, 250 μg, 260 μg, 270 μg, 275 μg, 280 μg, 290 μg, 300 μg, 310 μg, 320 μg, 325 μg, 330 μg, 340 μg, 350 μg, 360 μg, 370 μg, 375 μg, 400 μg, 425 μg, 450 μg, 500 μg, 550 μg, 600 μg, 650 μg, 700 μg, 750 μg, 800 μg, 850 μg, 900 μg, 950 μg, or 1000 μg of nucleic acid per 0.1 mM, 0.2 mM, 0.3 mM, 0.4 mM, 0.5 mM, 0.6 mM, 0.7 mM, 0.8 mM, 0.9 mM, 1 mM, 1.5 mM, 2 mM, 2.5 mM, 3 mM, 3.5 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 22 mM, 24 mM, 26 mM, 28 mM, 30 mM, 32 mM, 34 mM, 36 mM, 38 mM, or 40 mM lipids per 50, 100, 150, 200, 300, 400, 500, 600, 700, 800, 900, 1000, 1100, 1200, 1300, 1400, 1500, 2000, 2500, 3000, 3500, 4000, 4500, 5000, 5500, 6000, 7000, 8000, 9000, or 10,000 μl, as well as 15, 20, 25, or 50 ml, are mixed by adding the nucleic acid construct rapidly to the surface of the lipid (e.g., DOTAP: cholesterol) solution followed by mixing.

The nonviral vectors disclosed herein are typically of an average particle size of between about 40 nm and about 250 nm (e.g., 39 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 250 nm, 251 nm). In some embodiments, the average mean particle size of the nonviral vector is between about 300 and about 325 nm.

i. DOTAP: Cholesterol Liposomes

DOTAP: cholesterol liposomes are nanoparticle liposomal formulations composed of 1,2-bis(oleoyloxy)-3-(trimethyl ammonio) propane (DOTAP) and cholesterol. DOTAP: cholesterol liposomes form a stable structure and are efficient carriers of biologically active agents such as nucleic acid constructs. In embodiments, the liposomal formulation includes DOTAP in a concentration ranging from 1 to 8 millimolar (mM) (e.g., 1 mM, 2 to 7 mM, 3 to 6 mM, 4 to 5 mM, 8 mM). In embodiments, the liposomal formulation includes cholesterol or cholesterol derivative or cholesterol mixture in a concentration ranging from 0.1 to 8 mM (e.g., 0.1 mM, 0.2 to 1 mM, 2 to 7 mM, 3 to 6 mM, 4 to 5 mM, or 8 mM). In some embodiments of a nonviral vector, the DOTAP: cholesterol molar ratio is between about 3:1 and about 1:3 (e.g., about 3.1:1, about 3:1, about 2.5:1, about 2:1, about 1.5:1, about 1:1, about 1:1.5, about 1:2, about 1:2.5, about 1:3, or about 1:3.1). Methods of making DOTAP: cholesterol liposomes are known in the art. For example, extrusion, microfluidization, reverse phase evaporation, sonication, solvent (e.g., ethanol) injection, detergent dialysis, ether injection, and dehydration/rehydration may be utilized.

ii. Extrusion Techniques

The DOTAP: cholesterol liposomes described herein may be prepared, for example, by an extrusion method including the steps of heating, sonicating, and sequential extrusion of the lipids through filters of decreasing pore size, thereby resulting in the formation of small, stable liposome structures. In such methods, the production of liposomes often is accomplished by sonication or serial extrusion of liposomal mixtures after (i) reverse phase evaporation (ii) dehydration-rehydration (iii) detergent dialysis and (iv) thin film hydration. Methods of producing liposomes via extrusion are described in Templeton et al. (Nat. Biotechnol., 1997 15 (7): 647-52) and U.S. Pat. No. 10,293,056. In these methods, DNA: lipid complexes are prepared by diluting a given nucleic acid and lipids in 5% dextrose in water to obtain an appropriate concentration of nucleic acid and lipids in an isotonic solution. For example, DOTAP (cationic lipid) is mixed with cholesterol (neutral lipid) at about equimolar concentrations. This mixture of powdered lipids is then dissolved with a solvent such as chloroform. The lipid solution is dried to a thin film at 30° C. for 30 minutes (using, e.g., a rotary evaporator). The thin film is further freeze dried under vacuum for 15 minutes. The film is hydrated with water containing 5% dextrose (w/v) to give a final concentration of about 20 mM DOTAP and about 20 mM cholesterol. The hydrated lipid film is rotated in a 50° C. water bath for 45 minutes and then at 375° C. for an additional 10 minutes. The mixture is left standing at room temperature overnight. The following day the mixture is sonicated for 5-8 minutes at 50° C. The sonicated mixture is transferred to a new vessel and is heated for 10 minutes at 50° C. This mixture is sequentially extruded through filters (e.g., syringe filters) of decreasing pore size (e.g., 1 μm, 0.45 μm, 0.2 μm, and 0.1 μm). The 0.2 μm and 0.1 μm filters can be, e.g., Whatman Anotop filters (Cat. #: 6809-2122 or equivalent). The filtrate can be stored at, e.g., 4° C. or lower under argon or other inert gas.

iii. Microfluidization Techniques

In other embodiments, the DOTAP: cholesterol liposomes are produced using a microfluidization method. Microfluidization can be used when consistently small (40 to 200 nm) and relatively uniform aggregates are desired. Large scale production of DOTAP: cholesterol liposomes by microfluidization are known in the art. Methods of manufacturing liposomes using microfluidization are described, for example, in U.S. patent application Ser. No. 16/098,619. In certain microfluidization methods, the liposomal suspension is pumped at high velocity through an inlet that is divided into two streams and progressively bifurcates. These streams eventually collide within an interaction chamber leading to the formation of smaller particles due to turbulence and pressure. Generally, in microfluidization methods, DOTAP: cholesterol liposomes are formed by a quick increase in polarity of the environment induced by rapid mixing of the two miscible phases. This rapid mixing induces supersaturation of lipid molecules which leads to the self-assembly of DOTAP: cholesterol liposomes. Microfluidic mixing methods may include: microfluidic mixing using a staggered herringbone mixer (SHM), in-line T-junction mixing, and microfluidic hydrodynamic mixing (MHF). MHF is a continuous-flow technique where, in the case of liposome production, lipids dissolved in an organic solvent are hydrodynamically focused using an aqueous phase. In T-junction mixing, rapid mixing occurs when the two input streams in the T-junction collide, resulting in a turbulent output flow. SHM is microfluidic mixing by chaotic advection. Similar to other microfluidic techniques, the main characteristic is controlled millisecond mixing of two miscible phases, for example, ethanol and an aqueous buffer. The structure of the SHM allows efficient wrapping of the two fluids around each other resulting in an exponential enlargement of the interface between the fluids ensuring rapid mixing. In embodiments, a post-filtration step may be completed to reduce visible particles. In such embodiments, particles greater than 1 μm may be filtered out.

### III. Methods of Making Nonviral Vectors

Once manufactured, DOTAP: cholesterol liposomes can be used to encapsulate nucleic acids (e.g., a nucleic acid construct as described herein) resulting in the nonviral vectors described herein. In some embodiments, a nonviral vector is prepared by diluting nucleic acid constructs and lipids (DOTAP: cholesterol) in 5% dextrose in water to obtain an appropriate concentration of nucleic acid constructs and lipids (DOTAP: cholesterol). The nucleic acid constructs can be added to the DOTAP: cholesterol liposomes in a range of concentrations as indicated above. For example, equal volumes of nucleic acid construct and DOTAP: cholesterol, at a concentration to obtain about 100 μg of nucleic acid construct/about 0.1 to 4 mM lipids/about 100 μl, can be mixed by adding the nucleic acid construct rapidly to the DOTAP: cholesterol solution followed by rapid mixing.

In other methods, nonviral vectors can be produced using the heating, sonicating, and sequential extrusion methods described above. In some embodiments, nonviral vectors are produced using the microfluidization methods described above.

Once nonviral vectors are produced, they can be characterized using any suitable method. For example, mean particle size can be determined by dynamic light scattering using a particle size analyzer (e.g., a Malvern Zetasizer or Coulter N4 particle size analyzer).

### IV. Viral Vectors

The term “viral vector” is used herein to refers to a recombinant viral vector for delivering genetic material (e.g., a polynucleotide sequence encoding a NPRL2 protein) into a cell. A recombinant viral vector comprises capsid or envelope proteins and a recombinant viral genome, which is a nucleic acid construct comprising components derived from a viral genome (e.g., AAV) and heterologous polynucleotide sequences (e.g., a polynucleotide sequence encoding a NPRL2 protein or other therapeutic nucleic acid expression cassette). Examples of viral vectors include, but are not limited to, AAV vectors, retroviral vectors, lentiviral vectors, adenoviral vectors, herpesvirus vectors, alphavirus vectors, and the like.

A “recombinant AAV vector” or “rAAV vector” comprises a rAAV genome derived from the wild type genome of AAV. Typically, for AAV, one or both inverted terminal repeat (ITR) sequences of the wild type AAV genome are retained in the rAAV vector. A recombinant viral genome can be packaged into a virus (also referred to herein as a “particle” or “virion”) for subsequent infection (transformation) of a cell, ex vivo, in vitro or in vivo. Where a rAAV genome is encapsidated or packaged into an AAV particle, the particle can be referred to as a “rAAV.” Such particles or virions include proteins that encapsidate or package the viral genome. Particular examples include viral envelope proteins, and in the case of AAV, capsid proteins (VP1, VP2, VP3). As used herein, the term “serotype” refers to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Recombinant AAV vectors include AAV1, AAV2, AAV3, AAV4, AAV5, AAV6, AAV7, AAV8, AAV9, AAV10, AAV11, Rh10, Rh74 or AAV-218, and variants thereof. Examples of rAAV can include capsid proteins of any of AAV1, AAV2, AAV3, AAV4, AAV5, AAV6, AAV7, AAV8, AAV9, AAV10, AAV11, Rh10, Rh74 or AAV-218, or a capsid variant of AAV1, AAV2, AAV3, AAV4, AAV5, AAV6, AAV7, AAV8, AAV9, AAV10, AAV11, Rh10, Rh74 or AAV-218, or a capsid variant of AAV1, AAV2, AAV3, AAV4, AAV5, AAV6, AAV7, AAV8, AAV9, AAV10, AAV11, Rh10, Rh74 or AAV-218. Particular capsid variants include a capsid sequence with an amino acid substitution, deletion or insertion/addition.

A rAAV vector can comprise a genome derived from an AAV serotype distinct from the AAV serotype of one or more of the capsid proteins that package the recombinant viral genome. rAAV particles (vectors) can include one or more capsid proteins from a different serotype, a mixture of serotypes, or hybrids or chimeras of different serotypes, such as a VP1, VP2 or VP3 capsid protein of AAV1, AAV2, AAV3, AAV4, AAV5, AAV6, AAV7, AAV8, AAV9, AAV10, AAV11, Rh10, Rh74 or AAV-218 serotype. In some embodiments, an AAV serotype having a specific tissue tropism is used. rAAV can be produced using any suitable methods. Methods for large-scale production of rAAV are known and are described in, e.g., Urabe M. J. (2006) Virol. 80:1874-1885; Kotin R. M. (2011) Hum. Mol. Genet. 20: R2-6; Kohlbrenner E. et al. (2005) Mol. Ther. 12:1217-1225; Mietzsch M. (2014) Hum. Gene Ther. 25:212-222; and U.S. Pat. Nos. 6,436,392, 7,241,447, and 8,236,557.

### V. Compositions/Pharmaceutical Formulations

Compositions including the nucleic acid constructs, nonviral vectors, and viral vectors are described herein. In some embodiments, the composition includes a nonviral vector as described herein and dextrose, e.g., about 5% dextrose in water or saline. In other embodiments, the composition includes a nonviral vector as described herein and about 0.9% (e.g., 0.8%, 0.9%, 1.0%, etc.) sodium chloride. In additional embodiments, the composition includes a nonviral vector comprising a nucleic acid construct described herein and a combination of about 5% dextrose and about 0.9% sodium chloride.

The compositions, nucleic acid constructs, nonviral vectors and viral vectors described herein may be administered to mammals (e.g., rodents, humans, nonhuman primates, canines, felines, ovines, bovines) in a suitable formulation according to conventional pharmaceutical practice (see, e.g., Remington: The Science and Practice of Pharmacy (20th ed.), ed. A. R. Gennaro, Lippincott Williams & Wilkins, (2000) and Encyclopedia of Pharmaceutical Technology, eds. J. Swarbrick and J. C. Boylan, Marcel Dekker, New York (1988-1999)). A description of exemplary pharmaceutically acceptable carriers and diluents, as well as pharmaceutical formulations, can be found in Remington. Other substances may be added to the compositions to stabilize and/or preserve the compositions. As used herein the terms “pharmaceutically acceptable” means a biologically acceptable formulation, gaseous, liquid or solid, or mixture thereof, which is suitable for one or more routes of administration, in vivo delivery or contact. A pharmaceutically acceptable excipient is a material that is not biologically or otherwise undesirable, e.g., the material may be administered to a subject without causing substantial undesirable biological effects. In embodiments, the composition may comprise components that are generally regarded as safe.

The compositions described herein may be in a form suitable for sterile injection or infusion. To prepare such a composition, the active therapeutic(s) (e.g., nonviral vector) are dissolved or suspended in a parenterally acceptable liquid vehicle. Among acceptable vehicles, diluents and solvents that may be employed are water; water adjusted to a suitable pH by addition of an appropriate amount of a pH modifier (e.g., acid or base) or a suitable buffer; Ringer's solution; isotonic sodium chloride solution; and dextrose solution. For example, in one embodiment, the vectors may be administered over 0.5 to several hours by infusion with a pharmaceutically acceptable diluent such as 5% dextrose in water, Ringer's, and/or 0.9% NaCl. The aqueous formulation may also contain one or more preservatives (e.g., methyl, ethyl or n-propyl p-hydroxybenzoate). In cases where one of the therapeutics is only sparingly or slightly soluble in water, a dissolution enhancing or solubilizing agent can be added, or the solvent may include 10-60% w/w of propylene glycol or the like.

In other embodiments, the compositions described herein may be in a form suitable for intranasal administration. In one embodiment, the intranasal formulation is an aqueous formulation including a nucleic acid construct, nonviral vector or composition as described herein, a pH modifying agent, and a thickening agent. In the intranasal formulation, the pH modifying agent may provide or adjust the pH of the formulation to a suitable pH, e.g., a pH that assists in solubilizing an active agent in solution. In some embodiments, the intranasal formulation is administered as a stable intranasal spray that provides sufficient residence time on the nasal mucosa to allow trans-nasal absorption of the active agent(s). The thickening agent of the intranasal formulations described herein may modify the viscosity of the formulation to provide improved adherence of the formulation to the nasal mucosa without adversely affecting the ease of administration as an intranasal spray. The thickening agent may additionally increase the residence time of the formulation on the nasal mucosa, reduce loss of the formulation via mucociliary clearance of the nasal passages and/or improve the trans-nasal absorption. Such intranasal formulations may provide a sustained or controlled release of a nonviral vector as described herein.

The nucleic acid constructs, nonviral vectors, viral vectors and compositions described herein are preferably administered to a mammal (e.g., human) in a therapeutically effective amount. By the phrases “therapeutically effective amount”, “effective amount” and “effective dosage” is meant an amount sufficient to produce a therapeutically (e.g., clinically) desirable result; for example, the result can include increasing or restoring NPRL2 expression/signaling to NPRL2-deficient cancer cells, inducing apoptosis of cancer cells, decreasing tumor size, eliminating a tumor, or preventing or reducing metastasis in a subject. Dosage for a subject may depend on multiple factors, including the subject's size, body surface area, creatine clearance, age, the particular composition to be administered, time and route of administration, general health, and other drugs being administered concurrently. A delivery dose of a nucleic acid construct, nonviral vector, viral vector or composition as described herein is determined based on preclinical efficacy and safety.

In some embodiments, a therapeutically effective amount of nonviral vector as described herein or a composition containing a therapeutically effective amount of the nonviral vector is injected intravenously. In other embodiments, a therapeutically effective amount of nonviral vector as described herein or a composition containing a therapeutically effective amount of the nonviral vector is administered intranasally. The nonviral vectors, viral vectors and compositions can be administered, for example, as a “unit dose.” A unit dose as used herein is defined as containing a predetermined quantity of the therapeutic agent calculated to produce the desired responses in association with its administration, i.e., the appropriate route and treatment regimen. A unit dose as described herein may be described in terms of nucleic acid mass (μg) of the nucleic acid construct in the lipid complex. Unit doses range from 1, 25, 50, 75, 100, 125, 150, 175, 200, 225, 250, 300, 400, 500, 600, 700, 800, 900, 1000 μg and higher.

### VI. Methods of Treatment

Methods of treating cancer in a human subject are described herein. As used herein, the term “treating cancer” means administration of a therapeutic agent (e.g., nonviral vectors as described herein) to a patient having cancer with the purpose to cure, heal, alleviate, relieve, alter, remedy, ameliorate, improve or affect the disease, one or more symptoms of the disease, or predisposition toward disease. The treatment methods described herein inhibit, decrease or reduce one or more adverse (e.g., physical) symptoms, disorders, illnesses, diseases or complications caused by or associated with cancer, including for example, increasing or restoring NPRL2 to NPRL2-deficient cancer cells, inducing apoptosis of cancer cells, decreasing tumor size or eliminating a tumor in a subject, and/or reducing or preventing metastasis. Methods of treating cancer generally include increasing or restoring NPRL2 signaling/expression to cancer cells that have reduced NPRL2 levels or inhibition of NPRL2 signaling (e.g., colon cancer, pancreatic cancer, breast cancer, melanoma, osteosarcoma, leukemia, neuroblastoma, lung cancer, prostate cancer, renal cancer, or rectal cancer cells in a human subject). In one embodiment of a method of treating cancer, a composition including a nucleic acid construct as described herein is administered to a human subject in need thereof. In another embodiment of a method of treating cancer, a composition including a vector as described herein is administered to a human subject in need thereof. In a further embodiment of a method of treating cancer, a composition comprising a nonviral vector described herein is administered to a human subject in need thereof.

In embodiments, the disclosure provides a method for generating or augmenting an anti-tumor immune response in a human subject, comprising administering to the human subject in need thereof a pharmaceutical composition comprising a nucleic acid construct disclosed herein that expresses NPRL2 (e.g., from the codon optimized NPRL2 coding sequence of claim 1). In embodiments, the method for generating or augmenting an anti-tumor therapeutic immune response comprises administering to the human subject in need thereof a pharmaceutical composition comprising the nonviral vector disclosed herein (e.g., DOTAP/cholesterol liposomes with a NPRL2 expression construct, such as SEQ ID NO: 8). In embodiments, the method for generating or augmenting an anti-tumor immune response comprises administering to the human subject in need thereof the pharmaceutical compositions disclosed herein.

Any suitable methods of administering nucleic acid constructs, nonviral vectors, viral vectors, and compositions to a subject in need thereof may be used. In these methods, the nucleic acid constructs, nonviral vectors, viral vectors and compositions can be administered to the human subject by any suitable route. In some embodiments, for example, they are administered intravenously (IV). If administered via IV injection, the nucleic acid constructs, nonviral vectors, and compositions may be administered in a single bolus, multiple injections, or by continuous infusion (e.g., intravenously, pump infusion). In other embodiments, for example, they are administered intranasally. The nucleic acid constructs, nonviral vectors, and compositions can be administered to the human subject once (at one time point), or more than one time (e.g., two times, three times, four times, five times, six times, seven times, eight times, nine times, 10 times, etc.), i.e., at multiple time points. When the compositions are administered multiple times, the administrations may be separated by one day, three days, one week, two weeks, three weeks, one month, two months, or six months.

Some methods of treatment described herein are combination therapies that include administering to the human subject a nucleic acid construct, a nonviral vector, a viral vector, or a composition as described herein, and a second anti-cancer therapy. In embodiments the second anti-cancer therapy is radiation therapy. In embodiments the second anti-cancer therapy is chemotherapy, including, but not limited to, an alkylating agent (e.g., a platin-including carboplatin, cisplatin, or oxaliplatin-cyclophosphamide, melphalan, and temozolomide), an antimetabolite (e.g., 5-fluorouracil (5-FU), 6-mercaptopurine, cytarabine, gemcitabine, and methotrexate), an antitumor antibiotic (e.g., actinomycin-D, bleomycin, daunorubicin, and doxorubicin), and topoisomerase inhibitors (e.g., etoposide, irinotecan, teniposide, and topotecan). Another example of a second anti-cancer therapy is a checkpoint inhibitor. Use of checkpoint inhibitors as immunotherapy for treating cancer is known in the art (see U.S. patent application Ser. Nos. 15/536,718; 15/216,585; 15/648,423; 16/144,549). Examples of checkpoint inhibitors include PD-L1 inhibitors and PD-1 inhibitors such as pembrolizumab, Bavencio® (avelumab) and Tecentriq® (atezolizumab). Other examples of checkpoint inhibitors include Keytruda® (pembrolizumab) and Opdivo® (nivolumab). A further example of a second anti-cancer therapy is a BRAF inhibitor such as encorafenib. Another example of a second anti-cancer therapy is an EGFR inhibitor. Examples of EGFR inhibitors include cetuximab, osimertinib, Tarceva® (erlotinib), and nivolumab. In embodiments, the second anti-cancer therapy is a KRAS inhibitor. In embodiments, the second anti-cancer therapy is second nucleic acid construct. In embodiments, the second nucleic acid construct is a construct that encodes a tumor suppressor gene. For example, the second anti-cancer therapy may be a nucleic acid construct that encodes TUSC2. In embodiments of a combination therapy as described herein, the nucleic acid constructs, nonviral vectors, viral vectors or compositions are administered to the human subject before the second anti-cancer therapy is administered to the human subject (i.e., at two different time points). In another embodiment, the nucleic acid constructs, nonviral vectors, viral vectors and compositions are administered to the human subject at the same time that (concurrently with) the second anti-cancer therapy is administered. In another embodiment, the nucleic acid constructs, nonviral vectors, viral vectors and compositions are administered to the human subject after the second anti-cancer therapy is administered to the human subject (i.e., at two different time points). In some embodiments, a composition as described herein can include a nucleic acid construct, nonviral vector, or viral vector as described herein and a second anti-cancer therapy (e.g., a checkpoint inhibitor, a BRAF inhibitor, an EGFR inhibitor, etc.), i.e., admixed in the same injection or infusion volume.

### VII. Human Subjects

The terms “patient,” “subject,” and “individual” are used interchangeably herein, and mean a mammalian (e.g., human) subject in need of treatment with a vector comprising a NPRL2 expression construct (e.g., for treatment of cancer). Human subjects suffering from cancer include individuals suffering from various types of cancers, such as colon cancer, pancreatic cancer, breast cancer, melanoma, osteosarcoma, rectal cancer, lung cancer (e.g., small cell or non-small cell lung cancer), leukemia, and neuroblastoma. In the methods described herein, the subject can be undergoing surgery for any reason, such as for removal of diseased tissue, and/or radiation treatment. For example, in some embodiments of the methods described herein, the subject is undergoing, or has undergone, surgical resection of a tumor. As another example, in some embodiments of the methods described herein, the subject is undergoing, or has undergone, radiation treatment. As another example, in some embodiments of the methods described herein, the subject is undergoing, or has undergone, chemotherapy. In some embodiments of the methods described herein, the subject is undergoing, or has undergone, surgery (e.g., resection of a tumor) and/or radiation treatment and/or chemotherapy.

i. Molecular Markers

In some methods of treating cancer in a subject, the method includes selecting a subject having a cancer for administration of a therapy as described herein (e.g., administration of a composition containing a nonviral vector disclosed herein). A subject may be selected for therapy based on the presence of one or more mutations or other molecular markers for cancer in the subject's cancer cells. For example, a patient's tumor may be screened for having one or more mutations associated with a particular type of cancer, or types of cancer, including, e.g., BRAF mutations found in melanoma and colorectal cancer. Thus, a subject having cancer cells with the BRAF V600E mutation can be selected for receiving a therapy as described herein. Molecular markers associated with colon cancer are listed below in Table 1.

TABLE 1
Gene mutations and molecular markers
associated with colon cancer.

| Diagnosed Incident Cases by Molecular | |
|---|---|
| Markers (N) | (%) |
| KRAS Wild Type (N) | 51.55% |
| KRAS Mutation (N) | 48.45% |
| MET Amplification (N) | 1.69% |
| MSI - dMMR (N) | 17.00% |
| HER2 Amplification (N) | 2.82% |
| NTRK Gene Fusion (N) | 0.16% |
| NRAS Wild Type (N) | 95.79% |
| NRAS Mutation (N) | 4.21% |
| BRAF V600E Wild Type (N) | 93.87% |
| BRAF V600E Mutation (N) | 6.13% |

Molecular markers are known for several other cancers. For example, several EGFR mutations are associated with non-small cell lung cancer (NSCLC), adenocarcinoma, and squamous cell carcinoma. These mutations include: exon 19 deletion, exon 21 L858R substitution, exon 20 T790M mutation, exon 19 deletion and T790M, and exon 21 (L858R) and T790M. Several KRAS mutations are associated with NSCLC, adenocarcinoma and squamous cell carcinoma, including G12C, G12D and G12V. Other mutations associated with NSCLC, adenocarcinoma and squamous cell carcinoma include mutations in ALK, MET exon 14, PIK3CA, BRAF (V600E) and ROS1. A human subject having one or more of any of these mutations can be selected for treatment with the compositions, nucleic acid constructs, nonviral vectors, and methods described herein.

In some embodiments, subjects with a cancer having microsatellite instability (MSI) are selected for treatment with the therapies described herein. MSI is an important factor in the occurrence and development of tumors (e.g., gastric cancer, colon cancer, breast cancer) and molecular marker for cancer. MSI tumors may be characterized by high MSI (MSI-H) or low MSI (MSI-L). In some embodiments, a tumor characterized by MSI contains cells with MSI-H. A cell with high MSI is typically a cell having MSI at a level higher than a reference value or a control cell, e.g., a non-cancerous cell of the same tissue type as the cancer. In embodiments, nucleic acid constructs, vectors, and compositions disclosed herein are administered to a patient having a cancer with MSI. In embodiments of a method for treating cancer in a human subject as described herein, a composition, nucleic acid construct, or nonviral vector as described herein is administered to a human subject having a tumor characterized by MSI alone, or in combination with administration of a checkpoint inhibitor (e.g., pembrolizumab, nivolumab, etc.).

In embodiments, subjects with a cancer that is deficient in mismatch repair (dMMR) are selected for treatment with the therapies described herein. MMR deficiency is most common in colorectal cancer, other types of gastrointestinal cancer, and endometrial cancer, but may also be found in cancers of the breast, prostate, bladder, and thyroid.

Human subjects having cancer cells with a particular mutation (e.g., BRAF V600E) and/or having MSI and/or dMMR can be treated with a monotherapy or a combination therapy as described herein. In one embodiment of a combination therapy as described herein, the second anti-cancer therapy is specific for a cancer associated with a particular mutation. For example, if a human subject has a BRAF V600E mutation, the second anti-cancer therapy may be a BRAF inhibitor (e.g., encorafenib), or may be a combination of drugs including, for example, a BRAF inhibitor, e.g., encorafenib, cetuximab, and/or Mektovi® (binimetinib). In another embodiment of a combination therapy as described herein, the second anti-cancer therapy is specific for a cancer with MSI. For example, if a human subject has an MSI tumor, the second anti-cancer therapy may be a checkpoint inhibitor such as pembrolizumab or nivolumab. As another example, if the human subject has cancer cells with a KRAS mutation (e.g. a G12C mutation), the second anti-cancer therapy may be a KRAS inhibitor (e.g. sotorasib). In yet another embodiment of a combination therapy as described herein, the second anti-cancer therapy is specific for colon cancer. For example, if a human subject has colon cancer, the second anti-cancer therapy may be a cyclin dependent kinase (CDK).

It is to be understood and expected that variations of the compositions of matter and methods herein disclosed can be made by one skilled in the art and it is intended that such modifications are to be included within the scope of the present disclosure. All references cited herein are hereby incorporated by reference in their entirety.

TABLE 2
Sequences

| SEQ ID NO: | Description | Sequence |
|---|---|---|
| 1 | NPRL2 codon | ATGGGATCTGGCTGCAGAATCGAGTGCATCTTCTTCAG |
| | optimized | CGAGTTTCACCCCACACTGGGCCCCAAGATCACCTACC |
| | (NPRL2co) | AGGTGCCAGAGGACTTCATCAGCCGCGAGCTGTTCGAT |
| | coding sequence | ACCGTGCAGGTCTACATCATCACCAAGCCTGAGCTGCA |
| | | GAACAAGCTGATCACCGTGACCGCCATGGAAAAGAAG |
| | | CTGATCGGCTGCCCCGTGTGCATCGAGCACAAGAAGTA |
| | | CAGCAGAAACGCCCTGCTGTTCAACCTGGGCTTCGTGT |
| | | GTGATGCCCAGGCCAAGACCTGTGCTCTGGAACCCATC |
| | | GTGAAGAAGCTGGCCGGCTACCTGACCACACTGGAAC |
| | | TGGAAAGCAGCTTCGTGTCCATGGAAGAGTCCAAGCA |
| | | GAAACTGGTGCCCATCATGACCATCCTGCTGGAAGAAC |
| | | TGAACGCCAGCGGCAGATGCACCCTGCCTATCGACGA |
| | | GAGCAACACCATCCACCTGAAAGTGATCGAGCAGCGG |
| | | CCCGATCCTCCTGTGGCTCAAGAGTATGATGTGCCCGT |
| | | GTTCACCAAGGACAAAGAGGATTTCTTCAACAGCCAGT |
| | | GGGACCTGACAACCCAGCAGATCCTGCCTTACATCGAC |
| | | GGCTTCCGGCACATCCAGAAGATTAGCGCCGAGGCCG |
| | | ACGTGGAACTGAACCTCGTTAGAATCGCCATCCAGAAC |
| | | CTGCTGTATTACGGCGTGGTCACCCTGGTGTCCATCCT |
| | | GCAGTACAGCAACGTGTACTGCCCCACACCTAAGGTGC |
| | | AGGACCTGGTGGACGACAAGTCTCTGCAAGAGGCCTG |
| | | CCTGAGCTACGTGACAAAGCAGGGACACAAGAGAGCC |
| | | AGCCTGCGGGACGTGTTCCAGCTGTACTGTTCTCTGAG |
| | | CCCTGGCACCACAGTGCGGGATCTGATTGGCAGACATC |
| | | CCCAGCAGCTGCAGCACGTGGACGAGAGAAAGCTGAT |
| | | CCAGTTCGGCCTGATGAAGAACCTGATCCGCAGACTGC |
| | | AGAAATACCCCGTGCGCGTGACCAGAGAGGAACAGTC |
| | | TCATCCCGCCAGACTGTACACCGGCTGCCACAGCTACG |
| | | ACGAGATTTGCTGCAAGACCGGCATGAGCTACCACGA |
| | | GCTGGATGAGCGGCTGGAAAACGACCCCAACATCATC |
| | | ATCTGTTGGAAGTGATGA |
| 2 | CMV promoter | CACCAAAATCAACGGGACTTTCCAAAATGTCGTAACA |
| | sequence | ACTCCGCCCCATTGACGCAAATGGGCGGTAGGCGTGTA |
| | | CGGTGGGAGGTCTATATAAGCAGAGCTCGTTTAGTGAA |
| | | CCGTCAG |
| 3 | CMV enhancer | CTAGTTATTAATAGTAATCAATTACGGGGTCATTAGTT |
| | sequence | CATAGCCCATATATGGAGTTCCGCGTTACATAACTTAC |
| | | GGTAAATGGCCCGCCTGGCTGACCGCCCAACGACCCCC |
| | | GCCCATTGACGTCAATAATGACGTATGTTCCCATAGTA |
| | | ACGCCAATAGGGACTTTCCATTGACGTCAATGGGTGGA |
| | | GTATTTACGGTAAACTGCCCACTTGGCAGTACATCAAG |
| | | TGTATCATATGCCAAGTACGCCCCCTATTGACGTCAAT |
| | | GACGGTAAATGGCCCGCCTGGCATTATGCCCAGTACAT |
| | | GACCTTATGGGACTTTCCTACTTGGCAGTACATCTACG |
| | | TATTAGTCATCGCTATTACCATGGTGATGCGGTTTTGG |
| | | CAGTACATCAATGGGCGTGGATAGCGGTTTGACTCACG |
| | | GGGATTTCCAAGTCTCCACCCCATTGACGTCAATGGGA |
| | | GTTTGTTTTGG |
| 4 | HTLV-I | CTCGCATCTCTCCTTCACGCGCCCGCCGCCCTACCTGA |
| | regulatory | GGCCGCCATCCACGCCGGTTGAGTCGCGTTCTGCCGCC |
| | sequence | TCCCGCCTGTGGTGCCTCCTGAACTGCGTCCGCCGTCT |
| | | AGGTAAGTTTAAAGCTCAGGTCGAGACCGGGCCTTTGT |
| | | CCGGCGCTCCCTTGGAGCCTACCTAGACTCAGCCGGCT |
| | | CTCCACGCTTTGCCTGACCCTGCTTGCTCAACTCTA |
| 5 | BGH PolyA | CTGTGCCTTCTAGTTGCCAGCCATCTGTTGTTTGCCCCT |
| | sequence | CCCCCGTGCCTTCCTTGACCCTGGAAGGTGCCACTCCC |
| | | ACTGTCCTTTCCTAATAAAATGAGGAAATTGCATCGCA |
| | | TTGTCTGAGTAGGTGTCATTCTATTCTGGGGGGTGGGG |
| | | TGGGGCAGGACAGCAAGGGGGAGGATTGGGAAGACA |
| | | ATAGCAGGCATGCTGGGGATGCGGTGGGCTCTATGG |
| 6 | ß-globin intron | GTTAACTTAATGAGACAGATAGAAACTGGTCTTGTAGA |
| | sequence | AACAGAGTAGTCGCCTGCTTTTCTGCCAGGTGCTGACT |
| | | TCTCTCCCCTGGGCTTTTTTCTTTTTCTCAG |
| 7 | Bacterial | CCGCCTAATGAGCGGGCTTTTTTTTGGCTTGTTGTCCAC |
| | backbone | AACCGTTAAACCTTAAAAGCTTTAAAAGCCTTATATAT |
| | sequence | TCTTTTTTTTCTTATAAAACTTAAAACCTTAGAGGCTAT |
| | | TTAAGTTGCTGATTTATATTAATTTTATTGTTCAAACAT |
| | | GAGAGCTTAGTACGTGAAACATGAGAGCTTAGTACGTT |
| | | AGCCATGAGAGCTTAGTACGTTAGCCATGAGGGTTTAG |
| | | TTCGTTAAACATGAGAGCTTAGTACGTTAAACATGAGA |
| | | GCTTAGTACGTACTATCAACAGGTTGAACTGCTGATCC |
| | | ACGTTGTGGTAGAATTGGTAAAGAGAGTCGTGTAAAA |
| | | TATCGAGTTCGCACATCTTGTTGTCTGATTATTGATTTT |
| | | TGGCGAAACCATTTGATCATATGACAAGATGTGTATCT |
| | | ACCTTAACTTAATGATTTTGATAAAAATCATTA |
| 8 | One embodiment | CCGCCTAATGAGCGGGCTTTTTTTTGGCTTGTTGTCCAC |
| | of the expression | AACCGTTAAACCTTAAAAGCTTTAAAAGCCTTATATAT |
| | vector | TCTTTTTTTTCTTATAAAACTTAAAACCTTAGAGGCTAT |
| | Bacterial plasmid | TTAAGTTGCTGATTTATATTAATTTTATTGTTCAAACAT |
| | backbone: 1-454 | GAGAGCTTAGTACGTGAAACATGAGAGCTTAGTACGTT |
| | R6K origin: | AGCCATGAGAGCTTAGTACGTTAGCCATGAGGGTTTAG |
| | 26-306 | TTCGTTAAACATGAGAGCTTAGTACGTTAAACATGAGA |
| | trpA terminator: | GCTTAGTACGTACTATCAACAGGTTGAACTGCTGATCC |
| | 2914-28 | ACGTTGTGGTAGAATTGGTAAAGAGAGTCGTGTAAAA |
| | RNA-OUT: 316-454 | TATCGAGTTCGCACATCTTGTTGTCTGATTATTGATTTT |
| | CMV promoter: | TGGCGAAACCATTTGATCATATGACAAGATGTGTATCT |
| | 934-1053 | ACCTTAACTTAATGATTTTGATAAAAATCATTAGGTAC |
| | CMV enhancer: | CCCGGCTCTAGTTATTAATAGTAATCAATTACGGGGTC |
| | 467-933 | ATTAGTTCATAGCCCATATATGGAGTTCCGCGTTACAT |
| | HTLV-IR: | AACTTACGGTAAATGGCCCGCCTGGCTGACCGCCCAAC |
| | 1125-1350 | GACCCCCGCCCATTGACGTCAATAATGACGTATGTTCC |
| | ß globin intron: | CATAGTAACGCCAATAGGGACTTTCCATTGACGTCAAT |
| | 1359-1465 | GGGTGGAGTATTTACGGTAAACTGCCCACTTGGCAGTA |
| | CMV-HLTV-IR | CATCAAGTGTATCATATGCCAAGTACGCCCCCTATTGA |
| | Exon 1: | CGTCAATGACGGTAAATGGCCCGCCTGGCATTATGCCC |
| | 1466-1514 | AGTACATGACCTTATGGGACTTTCCTACTTGGCAGTAC |
| | Splicing | ATCTACGTATTAGTCATCGCTATTACCATGGTGATGCG |
| | enhancer (3x | GTTTTGGCAGTACATCAATGGGCGTGGATAGCGGTTTG |
| | gaagaagac SR | ACTCACGGGGATTTCCAAGTCTCCACCCCATTGACGTC |
| | binding | AATGGGAGTTTGTTTTGGCACCAAAATCAACGGGACTT |
| | protein): | TCCAAAATGTCGTAACAACTCCGCCCCATTGACGCAAA |
| | 1474-1500 | TGGGCGGTAGGCGTGTACGGTGGGAGGTCTATATAAG |
| | Exon 2: | CAGAGCTCGTTTAGTGAACCGTCAGATCGCCTGGAGAC |
| | 1466-1514 | GCCATCCACGCTGTTTTGACCTCCATAGAAGACACCGG |
| | Kozak: | GACCGATCCAGCCTCCGCGGCTCGCATCTCTCCTTCAC |
| | 1515-1523 | GCGCCCGCCGCCCTACCTGAGGCCGCCATCCACGCCGG |
| | NPRL2co: | TTGAGTCGCGTTCTGCCGCCTCCCGCCTGTGGTGCCTCC |
| | 1521-2666 | TGAACTGCGTCCGCCGTCTAGGTAAGTTTAAAGCTCAG |
| | BGH polyA: | GTCGAGACCGGGCCTTTGTCCGGCGCTCCCTTGGAGCC |
| | 2673 . . . 2897 | TACCTAGACTCAGCCGGCTCTCCACGCTTTGCCTGACC |
| | | CTGCTTGCTCAACTCTAGTTCTCTCGTTAACTTAATGAG |
| | | ACAGATAGAAACTGGTCTTGTAGAAACAGAGTAGTCG |
| | | CCTGCTTTTCTGCCAGGTGCTGACTTCTCTCCCCTGGGC |
| | | TTTTTTCTTTTTCTCAGGTTGAAAAGAAGAAGACGAAG |
| | | AAGACGAAGAAGACAAACCGTCGTCGACGCCACCATG |
| | | GGATCTGGCTGCAGAATCGAGTGCATCTTCTTCAGCGA |
| | | GTTTCACCCCACACTGGGCCCCAAGATCACCTACCAGG |
| | | TGCCAGAGGACTTCATCAGCCGCGAGCTGTTCGATACC |
| | | GTGCAGGTCTACATCATCACCAAGCCTGAGCTGCAGAA |
| | | CAAGCTGATCACCGTGACCGCCATGGAAAAGAAGCTG |
| | | ATCGGCTGCCCCGTGTGCATCGAGCACAAGAAGTACA |
| | | GCAGAAACGCCCTGCTGTTCAACCTGGGCTTCGTGTGT |
| | | GATGCCCAGGCCAAGACCTGTGCTCTGGAACCCATCGT |
| | | GAAGAAGCTGGCCGGCTACCTGACCACACTGGAACTG |
| | | GAAAGCAGCTTCGTGTCCATGGAAGAGTCCAAGCAGA |
| | | AACTGGTGCCCATCATGACCATCCTGCTGGAAGAACTG |
| | | AACGCCAGCGGCAGATGCACCCTGCCTATCGACGAGA |
| | | GCAACACCATCCACCTGAAAGTGATCGAGCAGCGGCC |
| | | CGATCCTCCTGTGGCTCAAGAGTATGATGTGCCCGTGT |
| | | TCACCAAGGACAAAGAGGATTTCTTCAACAGCCAGTG |
| | | GGACCTGACAACCCAGCAGATCCTGCCTTACATCGACG |
| | | GCTTCCGGCACATCCAGAAGATTAGCGCCGAGGCCGA |
| | | CGTGGAACTGAACCTCGTTAGAATCGCCATCCAGAACC |
| | | TGCTGTATTACGGCGTGGTCACCCTGGTGTCCATCCTG |
| | | CAGTACAGCAACGTGTACTGCCCCACACCTAAGGTGCA |
| | | GGACCTGGTGGACGACAAGTCTCTGCAAGAGGCCTGC |
| | | CTGAGCTACGTGACAAAGCAGGGACACAAGAGAGCCA |
| | | GCCTGCGGGACGTGTTCCAGCTGTACTGTTCTCTGAGC |
| | | CCTGGCACCACAGTGCGGGATCTGATTGGCAGACATCC |
| | | CCAGCAGCTGCAGCACGTGGACGAGAGAAAGCTGATC |
| | | CAGTTCGGCCTGATGAAGAACCTGATCCGCAGACTGCA |
| | | GAAATACCCCGTGCGCGTGACCAGAGAGGAACAGTCT |
| | | CATCCCGCCAGACTGTACACCGGCTGCCACAGCTACGA |
| | | CGAGATTTGCTGCAAGACCGGCATGAGCTACCACGAG |
| | | CTGGATGAGCGGCTGGAAAACGACCCCAACATCATCA |
| | | TCTGTTGGAAGTGATGAGAATTCCTGTGCCTTCTAGTT |
| | | GCCAGCCATCTGTTGTTTGCCCCTCCCCCGTGCCTTCCT |
| | | TGACCCTGGAAGGTGCCACTCCCACTGTCCTTTCCTAA |
| | | TAAAATGAGGAAATTGCATCGCATTGTCTGAGTAGGTG |
| | | TCATTCTATTCTGGGGGGTGGGGTGGGGCAGGACAGC |
| | | AAGGGGGAGGATTGGGAAGACAATAGCAGGCATGCTG |
| | | GGGATGCGGTGGGCTCTATGGCCCGGGACGGCCGCTAGC |
| 9 | NPRL2 amino | MGSGCRIECIFFSEFHPTLGPKITYQVPEDFISRELFDTVQV |
| | acid sequence | YIITKPELQNKLITVTAMEKKLIGCPVCIEHKKYSRNALLF |
| | | NLGFVCDAQAKTCALEPIVKKLAGYLTTLELESSFVSMEE |
| | | SKQKLVPIMTILLEELNASGRCTLPIDESNTIHLKVIEQRPD |
| | | PPVAQEYDVPVFTKDKEDFFNSQWDLTTQQILPYIDGFRH |
| | | IQKISAEADVELNLVRIAIQNLLYYGVVTLVSILQYSNVYC |
| | | PTPKVQDLVDDKSLQEACLSYVTKQGHKRASLRDVFQL |
| | | YCSLSPGTTVRDLIGRHPQQLQHVDERKLIQFGLMKNLIR |
| | | RLQKYPVRVTREEQSHPARLYTGCHSYDEICCKTGMSYH |
| | | ELDERLENDPNIIICWK |

### EXAMPLES

### Example 1. Antitumor Effect of NPRL2 Expression in Syngeneic Mouse Lung Cancer Model

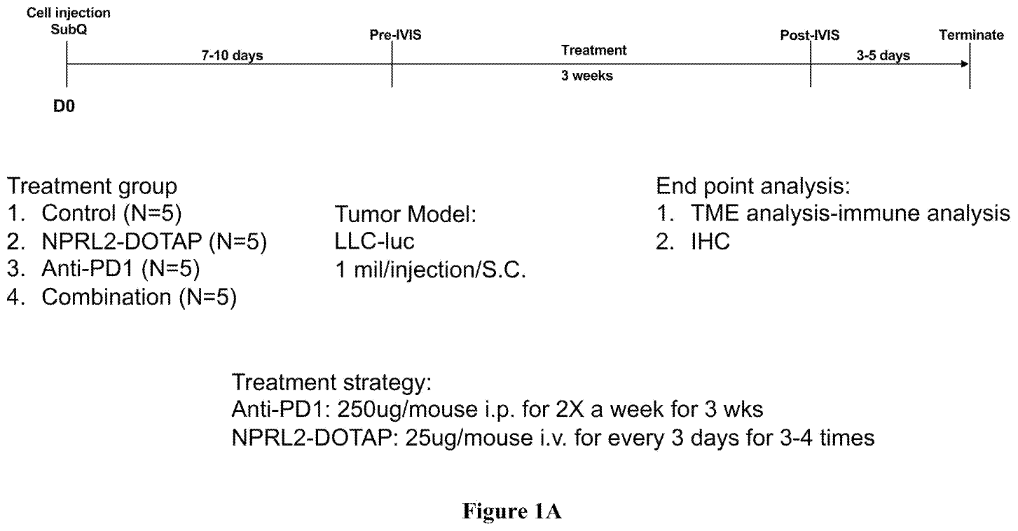

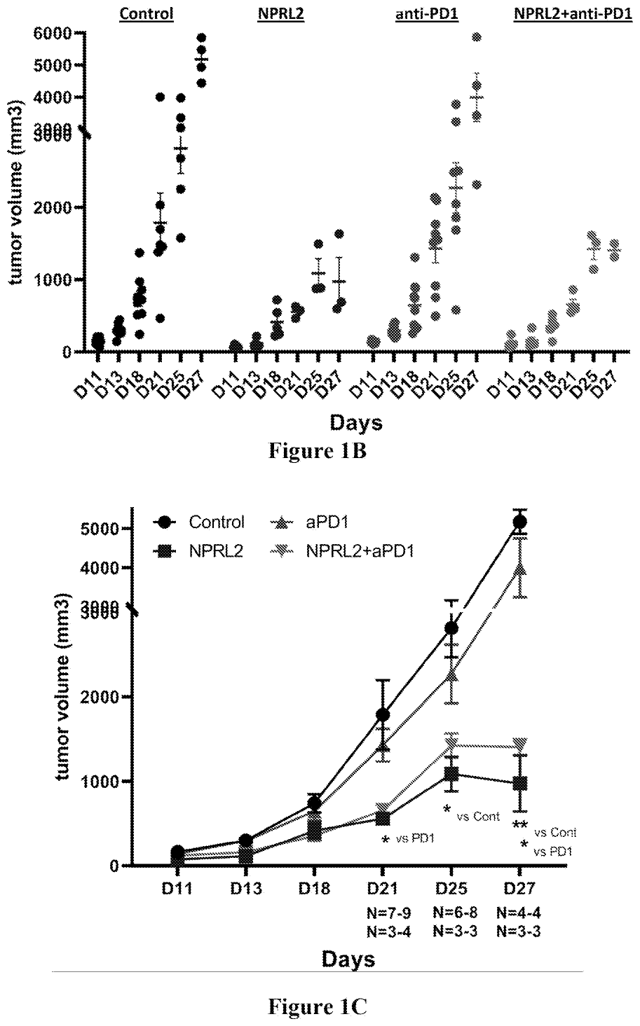

DOTAP-NPRL2 alone or in combination with anti-PD1 antibody was tested in a syngeneic mouse model with LLC2 (lung carcinoma) tumors that are KRAS mutant and anti-PD1 resistant by the protocol in FIG. 1A. DOTAP-NPRL2 was made by loading DOTAP/cholesterol nanovesicles with the NPRL2 expression construct (SEQ ID NO: 8). Control group was treated with nanovesicles loaded with empty vector.

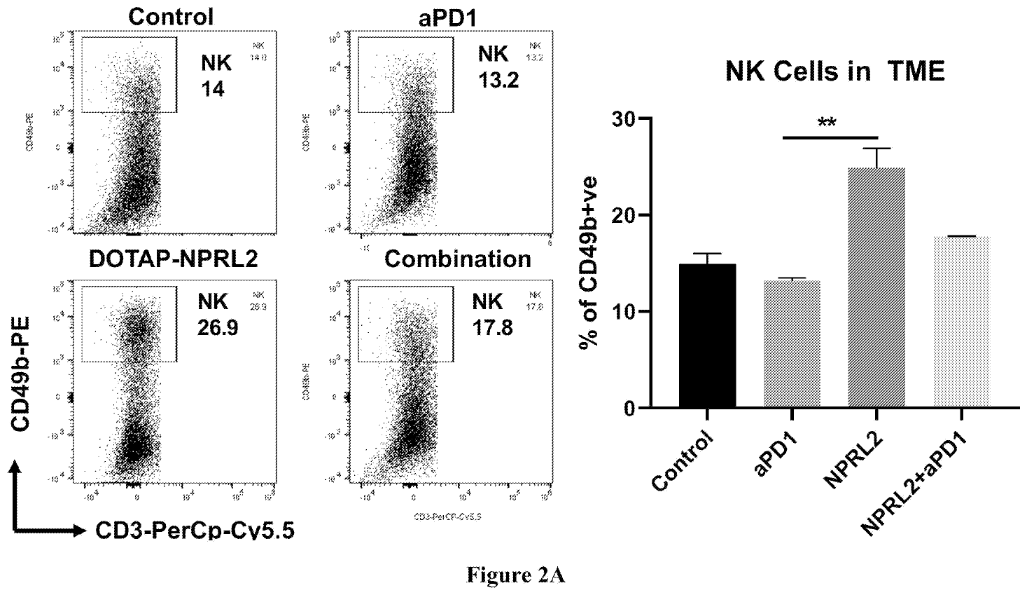

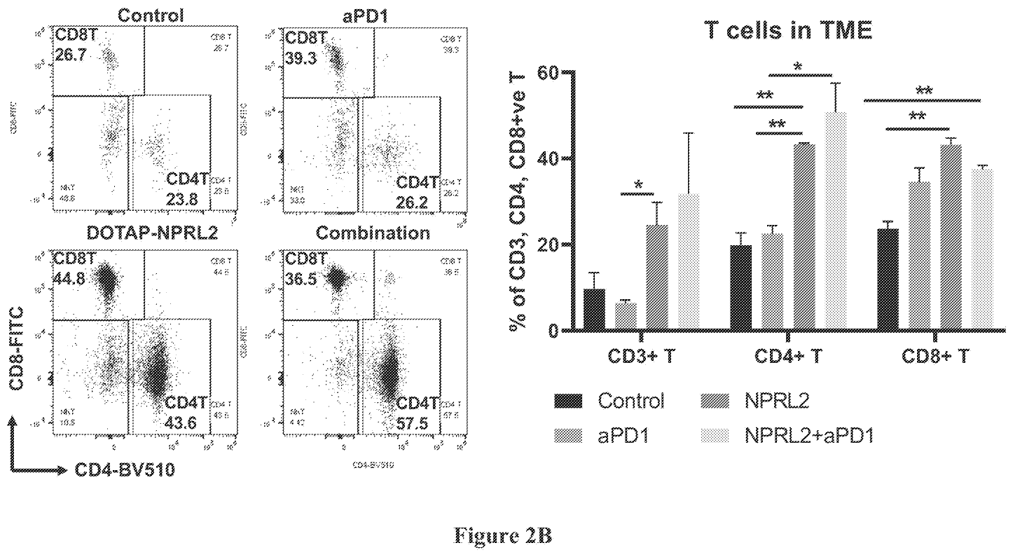

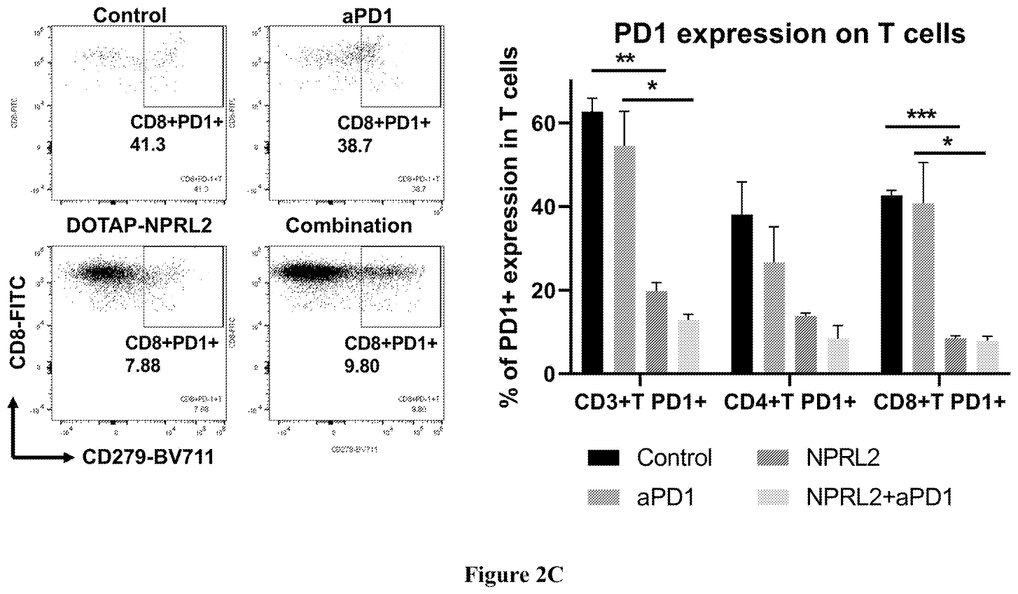

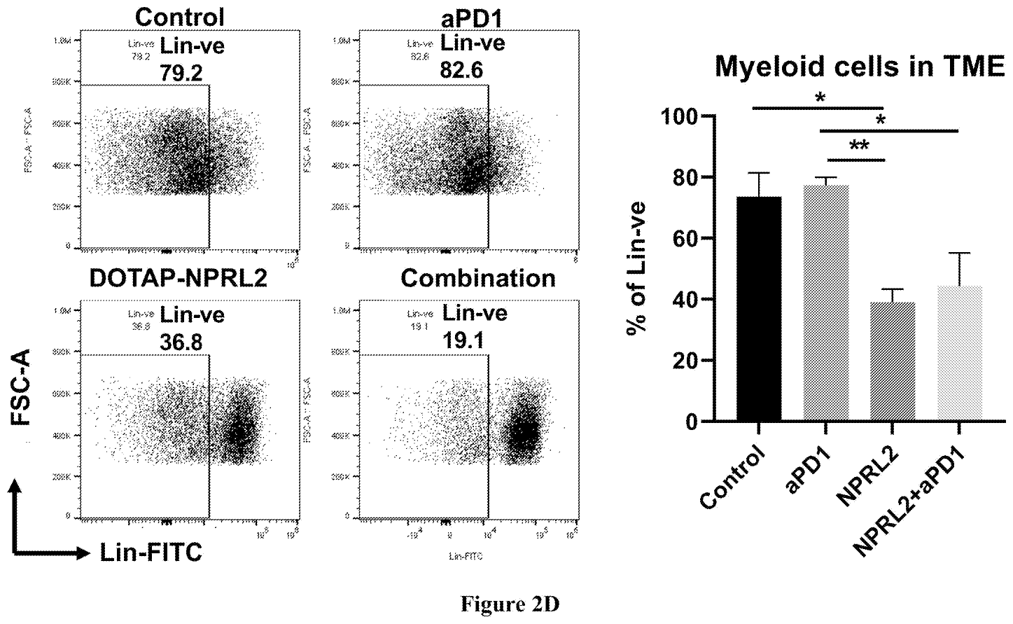

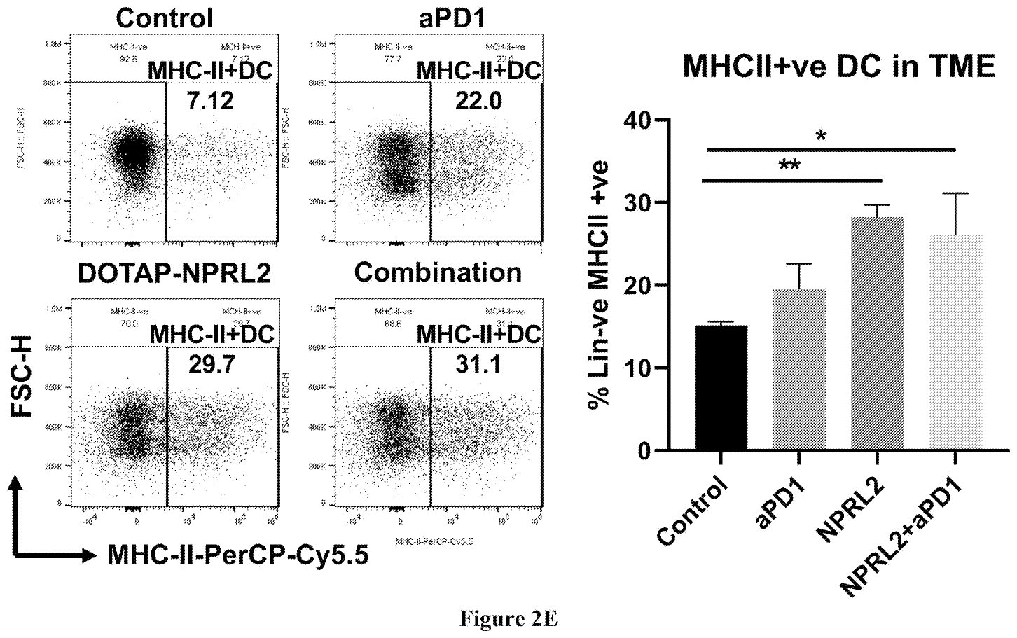

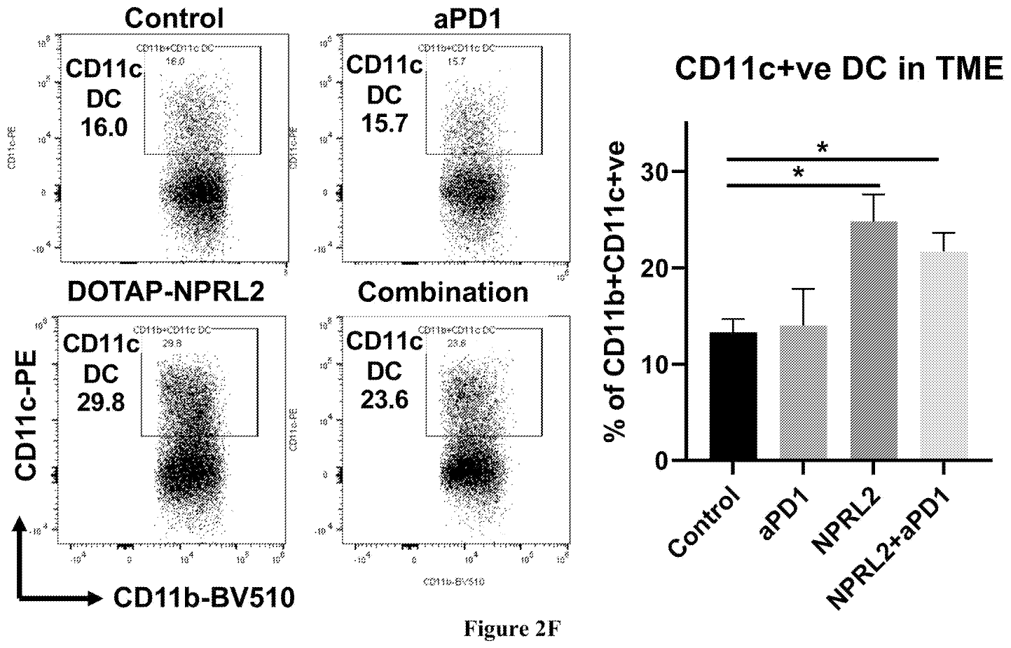

Consistent with the humanized mouse models discussed below, NPRL2 expression from the DOTAP-NPRL2 showed a significantly strong antitumor effect, whereas anti-PD1 was not effective in this model (FIGS. 1B and 1C). The antitumor effect of NPRL2 was correlated with the presence of innate immune cells including HLA-DR+ DC and CD11c DC and adaptive immune cells including TILs and NK cells in the tumor microenvironment (FIGS. 2A, 2B, 2E and 2F). In addition, NPRL2 treatment downregulated the myeloid and regulatory T cells (FIGS. 2C-2D).

### Example 2. Antitumor and Immune Effects of NPRL2 Expression in Humanized Mouse Models of NSCLC

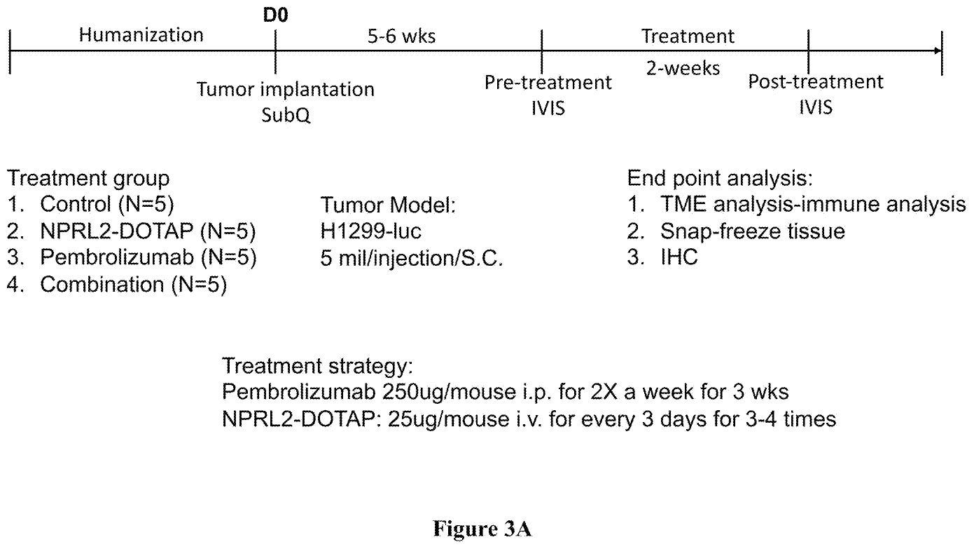

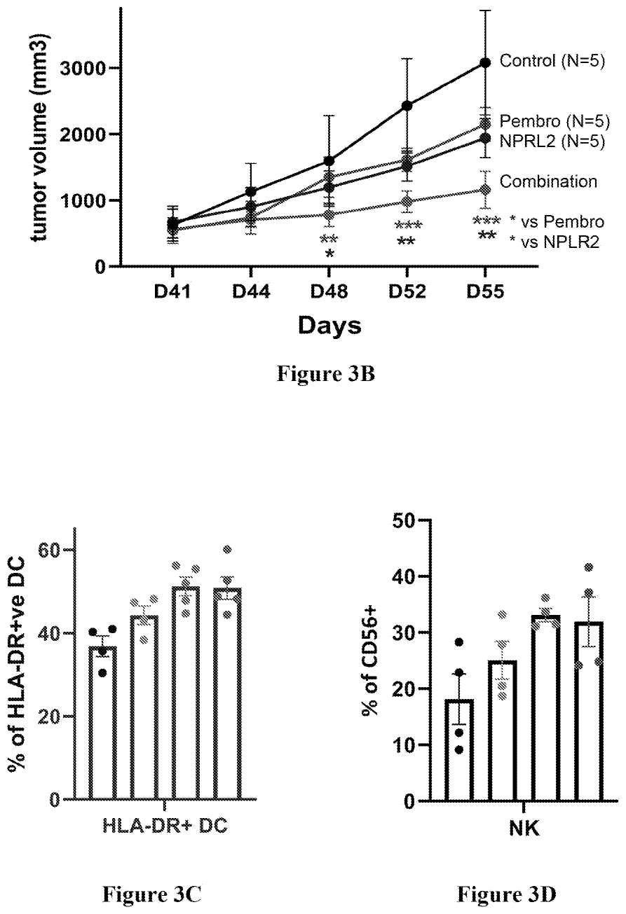

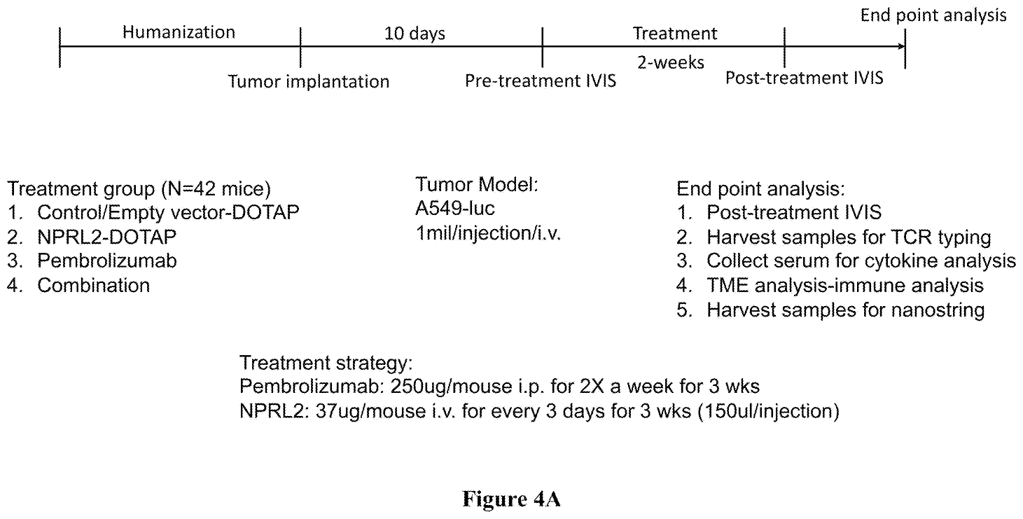

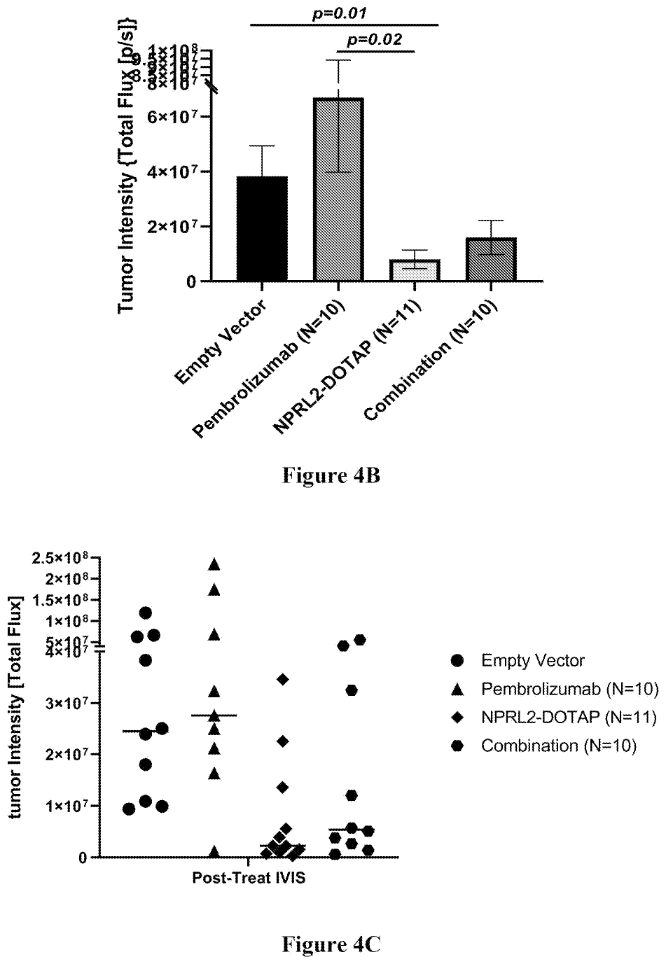

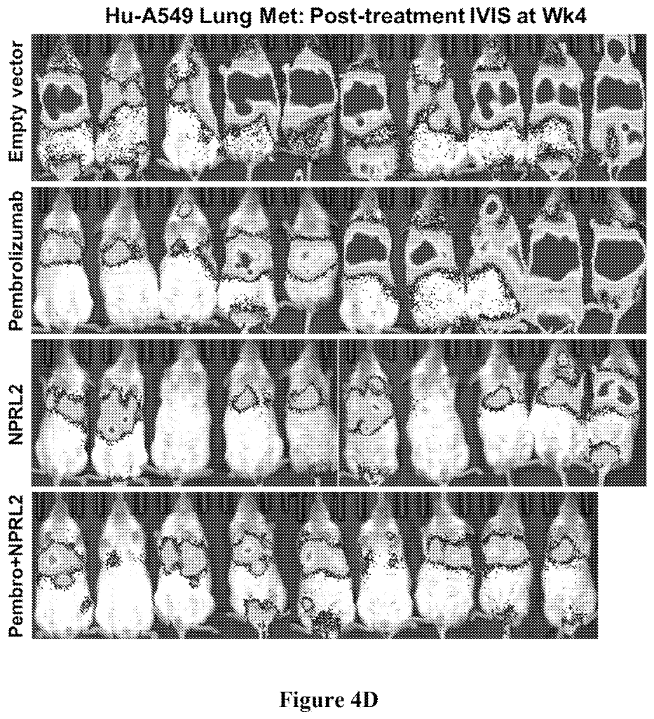

The antitumor immune responses to NPRL2 gene therapy was investigated on NSCLC tumors from subcutaneous implantation of H1299 or A549 cells in a humanized mouse model by the protocols in FIGS. 3A and 4A, respectively. Humanized mice were generated by transplanting fresh human cord blood derived CD34 stem cells into sub-lethally irradiated NSG mice. The level of engraftment of human CD45, CD3 T, CD19 B, NK cells was verified before tumor implantation. Mice harboring >25% human CD45 cells were considered humanized.

Humanized mice with H1299 tumors (KRAS wild type, anti-PD1 sensitive) showed significant reduction in tumor volume after treatment with DOTAP-NPRL2 with the combination of DOTAP-NPRL2 and anti-PD1 antibody showing a significant further reduction in tumor volume (FIG. 3B). Thus, a robust and synergistic antitumor effect was observed in the KRAS wild type, anti-PD1 sensitive H1299 tumors grown in humanized mice treated with NPRL2 and pembrolizumab (FIG. 3B). Cytotoxic T cells, NK cells, and HLA-DR+ DC were associated with the antitumor effect (see, e.g., FIGS. 3C and 3D).

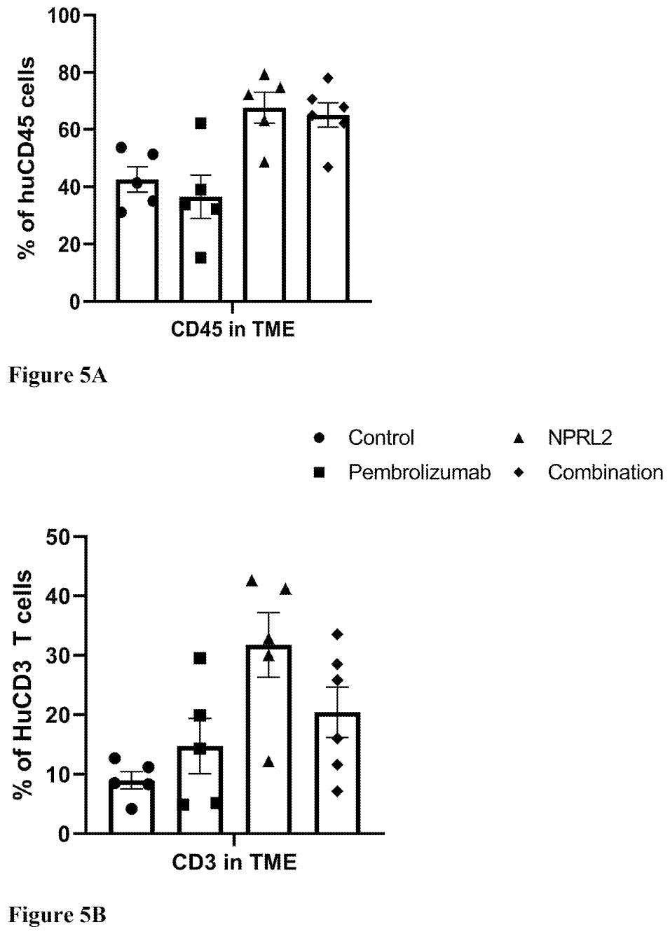

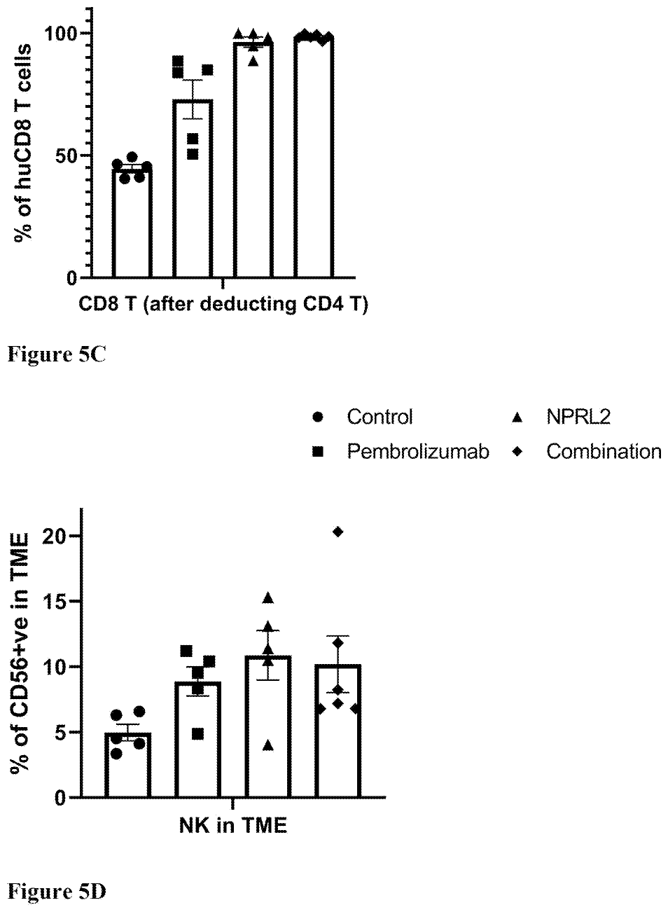

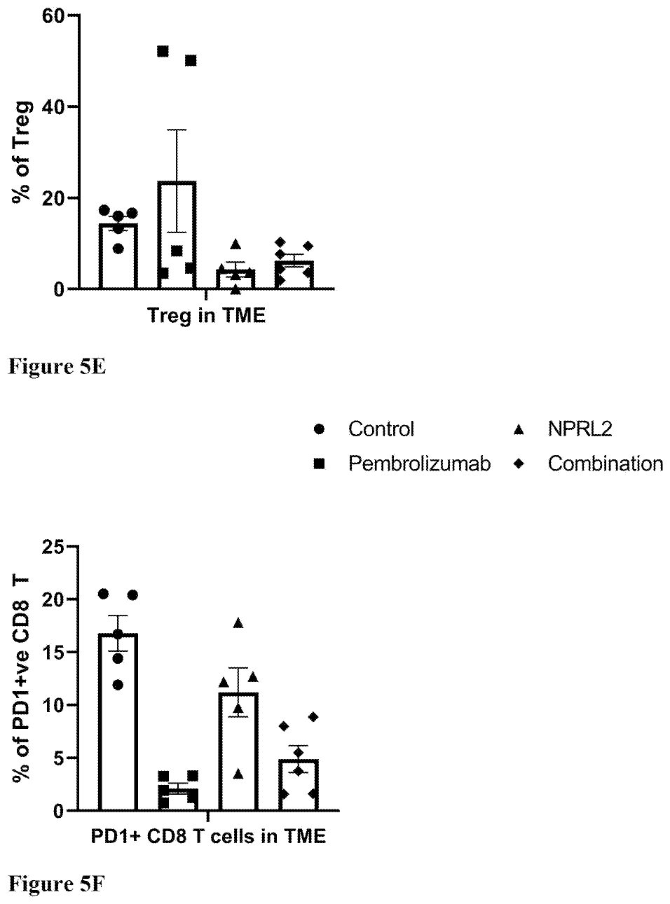

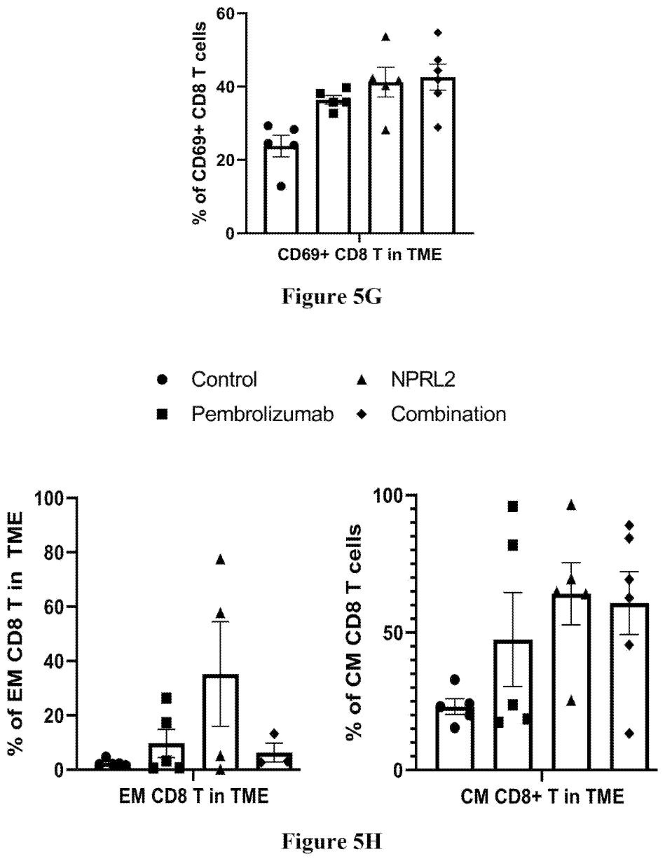

KRAS/STK11 mutant anti-PD1 resistant A549 NSCLC cells were injected intravenously into fully humanized NSG mice and developed lung metastasis. Metastases were treated with intravenous injection of NPRL2 gene loaded cationic lipid nanoparticles (DOTAP-NPRL2) with or without anti-PD1 antibody (pembrolizumab). Control group was treated with nanovesicles loaded with empty vector. A dramatic antitumor effect was mediated by NPRL2 treatment, whereas pembrolizumab alone was ineffective (FIGS. 4B, 4C and 4D). A significant antitumor effect was also found in non-humanized NSG mice, although the antitumor effect was greater in humanized mice suggesting that the immune response played a role in inducing antitumor activity. The antitumor effect of NPRL2 was associated with increased infiltration of human CD45, CD3 T, cytotoxic T, NK cells (FIGS. 5A-5D), and a decreased number of human regulatory T cells (Treg) in tumors (FIG. 5E).

PD1 expressing exhausted CD8 T cells were downregulated in both the NPRL2 and pembrolizumab groups (FIG. 5F). The number of activated T cells (CD69+CD8+T), effector memory (EM) and central memory (CM) CD8 T cells were significantly increased by NPRL2 treatment and NPRL2 induced antigen presenting HLA-DR+ve dendritic cells (FIGS. 5D, 5G, and 5H). When NPRL2 was combined with pembrolizumab, no synergistic antitumor effect was found in the KRAS/STK11 mutant anti-PD1 insensitive tumors. Taken together, these data suggest that the NPRL2 gene therapy disclosed herein induces antitumor activity on KRAS/STK11 mutant anti-PD1 resistant tumors through DC mediated antigen presentation and cytotoxic immune cell activation.

We claim:

1. A codon optimized polynucleotide sequence encoding a human NPRL2 protein, wherein the polynucleotide sequence comprises SEQ ID NO:1.

2. A nucleic acid construct comprising the polynucleotide sequence according to claim 1, further comprising a CMV promoter operably linked to the polynucleotide sequence encoding a human NPRL2 protein.

3. The nucleic acid construct of claim 2, wherein the CMV promoter comprises a sequence having greater than 90% sequence identity to SEQ ID NO: 2.

4. The nucleic acid construct of claim 3, wherein the CMV promoter comprises SEQ ID NO: 2.

5. The nucleic acid construct of any of claims 2-4, further comprising a CMV enhancer.

6. The nucleic acid construct of claim 5, wherein the CMV enhancer comprises a sequence having greater than 90% sequence identity to SEQ ID NO: 3.

7. The nucleic acid construct of claim 6, wherein the CMV enhancer comprises SEQ ID NO: 3.

8. The nucleic acid construct of any of claims 2-7, further comprising a HTLV-I regulatory sequence.

9. The nucleic acid construct of claim 8, wherein the HTLV-I regulatory sequence comprises a sequence having greater than 90% sequence identity to SEQ ID NO: 4.

10. The nucleic acid construct of claim 9, wherein the HTLV-I regulatory sequence comprises SEQ ID NO: 4.

11. The nucleic acid construct of any of claims 2-10, further comprising a bovine growth hormone polyadenylation (BGH polyA) sequence.

12. The nucleic acid construct of claim 11, wherein the BGH poly A sequence comprises a sequence having greater than 90% sequence identity to SEQ ID NO: 5.

13. The nucleic acid construct of claim 12, wherein the BGH polyA sequence comprises SEQ ID NO: 5.

14. The nucleic acid construct of any of claims 2-13, further comprising a splicing enhancer sequence.

15. The nucleic acid construct of any of claims 2-14, further comprising at least one intron.

16. The nucleic acid construct of claim 15, wherein the at least one intron is a β-globin intron.

17. The nucleic acid construct of claim 16, wherein the β-globin intron sequence comprises a sequence having greater than 90% sequence identity to SEQ ID NO: 6.

18. The nucleic acid construct of claim 17, wherein the β-globin intron sequence comprises SEQ ID NO: 6.

19. The nucleic acid construct of any of claims 2-18, further comprising a bacterial backbone sequence.

20. The nucleic acid construct of claim 19, wherein the bacterial backbone sequence comprises a sequence having greater than 90% sequence identity to SEQ ID NO: 7.

21. The nucleic acid construct of claim 20, wherein the bacterial backbone sequence comprises SEQ ID NO: 7.

22. The nucleic acid construct of claim 19-21, wherein the bacterial backbone sequence comprises a R6K origin sequence.

23. The nucleic acid construct of claim 19-22, wherein the bacterial backbone sequence comprises a selectable marker.

24. The nucleic acid construct of any of claims 2-23, comprising a sequence having greater than 90% sequence identity to SEQ ID NO: 8.

25. The nucleic acid construct of claim 24, comprising SEQ ID NO: 8.

26. The nucleic acid construct of any of claims 2-25, wherein the nucleic acid sequence encoding the NPRL2 protein encodes a protein comprising a sequence that is at least 90% identical to SEQ ID NO: 9.

27. The nucleic acid construct of any of claims 2-25, wherein the nucleic acid sequence encoding the NPRL2 protein encodes a protein comprising SEQ ID NO: 9

28. A nonviral vector comprising a nucleic acid construct according to any one of claims 2-27 and a DOTAP: cholesterol liposome.

29. The nonviral vector of claim 28, wherein the DOTAP: cholesterol ratio is between about 3:1 and about 1:3.

30. A pharmaceutical composition comprising the nonviral vector of claim 28 or claim 29 and a pharmaceutically acceptable excipient.

31. The pharmaceutical composition of claim 30, further comprising approximately 5% dextrose, 0.9% sodium chloride or a combination of both agents.

32. The pharmaceutical composition of claim 31, wherein the DOTAP: cholesterol liposome has a particle size range of about 40 to 250 nanometers.

33. A method for treating cancer in a human subject comprising administering to the human subject in need thereof a pharmaceutical composition comprising a nucleic acid construct according to any one of claims 2-27.

34. A method for treating cancer in a human subject comprising administering to the human subject in need thereof a pharmaceutical composition comprising the nonviral vector according to any one of claims 28-29.

35. A method for treating cancer in a human subject comprising administering to the human subject in need thereof a pharmaceutical composition according to any one of claims 31 to 32.

36. The method according to any one of claims 33 to 35, wherein the cancer is selected from the group consisting of: colon cancer, pancreatic cancer, breast cancer, melanoma, osteosarcoma, neuroblastoma, leukemia, lung cancer, renal cancer, and rectal cancer.

37. The method according to any one of claims 33 to 36, wherein the pharmaceutical composition is administered intravenously or intranasally.

38. The method according to any one of claims 33 to 37, further comprising administering a second anti-cancer therapy to the subject.

39. The method of claim 38, wherein the second anti-cancer therapy comprises at least one of: chemotherapy, radiation treatment, and surgery.

40. The method of claim 38, wherein the second anti-cancer therapy is a checkpoint inhibitor or a BRAF inhibitor.

41. The method of claim 38, wherein the second anti-cancer therapy is an EGFR inhibitor.

42. The method of claim 40, wherein the checkpoint inhibitor is pembrolizumab and the BRAF inhibitor is encorafenib.

43. The method of claim 41, wherein the EGFR inhibitor is cetuximab or nivolumab.

44. The method of claim 38, wherein the second anti-cancer therapy is a KRAS inhibitor.

45. A viral vector comprising a nucleic acid construct according to any of claims 2-27.

46. A method for treating cancer in a human subject comprising administering to the human subject in need thereof the viral vector of claim 45.

47. The viral vector of claim 46, wherein the viral vector is an Adeno-Associated Virus (AAV) viral vector.


SEQUENCE LISTING (9 sequences)

SEQ ID NO: 1 - DNA, 1146 bp, synthetic construct
  source 1..1146: mol_type = other DNA
atgggatctggctgcagaatcgagtgcatcttcttcagcgagtttcaccccacactgggccccaagatcacctaccaggtgccagaggacttcatcagccgcgagctgttcgataccgtgcaggtctacatcatcaccaagcctgagctgcagaacaagctgatcaccgtgaccgccatggaaaagaagctgatcggctgccccgtgtgcatcgagcacaagaagtacagcagaaacgccctgctgttcaacctgggcttcgtgtgtgatgcccaggccaagacctgtgctctggaacccatcgtgaagaagctggccggctacctgaccacactggaactggaaagcagcttcgtgtccatggaagagtccaagcagaaactggtgcccatcatgaccatcctgctggaagaactgaacgccagcggcagatgcaccctgcctatcgacgagagcaacaccatccacctgaaagtgatcgagcagcggcccgatcctcctgtggctcaagagtatgatgtgcccgtgttcaccaaggacaaagaggatttcttcaacagccagtgggacctgacaacccagcagatcctgccttacatcgacggcttccggcacatccagaagattagcgccgaggccgacgtggaactgaacctcgttagaatcgccatccagaacctgctgtattacggcgtggtcaccctggtgtccatcctgcagtacagcaacgtgtactgccccacacctaaggtgcaggacctggtggacgacaagtctctgcaagaggcctgcctgagctacgtgacaaagcagggacacaagagagccagcctgcgggacgtgttccagctgtactgttctctgagccctggcaccacagtgcgggatctgattggcagacatccccagcagctgcagcacgtggacgagagaaagctgatccagttcggcctgatgaagaacctgatccgcagactgcagaaataccccgtgcgcgtgaccagagaggaacagtctcatcccgccagactgtacaccggctgccacagctacgacgagatttgctgcaagaccggcatgagctaccacgagctggatgagcggctggaaaacgaccccaacatcatcatctgttggaagtgatga

SEQ ID NO: 2 - DNA, 120 bp, synthetic construct
  source 1..120: mol_type = other DNA
caccaaaatcaacgggactttccaaaatgtcgtaacaactccgccccattgacgcaaatgggcggtaggcgtgtacggtgggaggtctatataagcagagctcgtttagtgaaccgtcag

SEQ ID NO: 3 - DNA, 467 bp, synthetic construct
  source 1..467: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgg

SEQ ID NO: 4 - DNA, 226 bp, synthetic construct
  source 1..226: mol_type = other DNA
ctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactcta

SEQ ID NO: 5 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 6 - DNA, 107 bp, synthetic construct
  source 1..107: mol_type = other DNA
gttaacttaatgagacagatagaaactggtcttgtagaaacagagtagtcgcctgcttttctgccaggtgctgacttctctcccctgggcttttttctttttctcag

SEQ ID NO: 7 - DNA, 454 bp, synthetic construct
  source 1..454: mol_type = other DNA
ccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcatta

SEQ ID NO: 8 - DNA, 2915 bp, synthetic construct
  source 1..2915: mol_type = other DNA
ccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccccggctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcgtcgacgccaccatgggatctggctgcagaatcgagtgcatcttcttcagcgagtttcaccccacactgggccccaagatcacctaccaggtgccagaggacttcatcagccgcgagctgttcgataccgtgcaggtctacatcatcaccaagcctgagctgcagaacaagctgatcaccgtgaccgccatggaaaagaagctgatcggctgccccgtgtgcatcgagcacaagaagtacagcagaaacgccctgctgttcaacctgggcttcgtgtgtgatgcccaggccaagacctgtgctctggaacccatcgtgaagaagctggccggctacctgaccacactggaactggaaagcagcttcgtgtccatggaagagtccaagcagaaactggtgcccatcatgaccatcctgctggaagaactgaacgccagcggcagatgcaccctgcctatcgacgagagcaacaccatccacctgaaagtgatcgagcagcggcccgatcctcctgtggctcaagagtatgatgtgcccgtgttcaccaaggacaaagaggatttcttcaacagccagtgggacctgacaacccagcagatcctgccttacatcgacggcttccggcacatccagaagattagcgccgaggccgacgtggaactgaacctcgttagaatcgccatccagaacctgctgtattacggcgtggtcaccctggtgtccatcctgcagtacagcaacgtgtactgccccacacctaaggtgcaggacctggtggacgacaagtctctgcaagaggcctgcctgagctacgtgacaaagcagggacacaagagagccagcctgcgggacgtgttccagctgtactgttctctgagccctggcaccacagtgcgggatctgattggcagacatccccagcagctgcagcacgtggacgagagaaagctgatccagttcggcctgatgaagaacctgatccgcagactgcagaaataccccgtgcgcgtgaccagagaggaacagtctcatcccgccagactgtacaccggctgccacagctacgacgagatttgctgcaagaccggcatgagctaccacgagctggatgagcggctggaaaacgaccccaacatcatcatctgttggaagtgatgagaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgggacggccgctagc

SEQ ID NO: 9 - protein, 380 aa, synthetic construct
MGSGCRIECIFFSEFHPTLGPKITYQVPEDFISRELFDTVQVYIITKPELQNKLITVTAMEKKLIGCPVCIEHKKYSRNALLFNLGFVCDAQAKTCALEPIVKKLAGYLTTLELESSFVSMEESKQKLVPIMTILLEELNASGRCTLPIDESNTIHLKVIEQRPDPPVAQEYDVPVFTKDKEDFFNSQWDLTTQQILPYIDGFRHIQKISAEADVELNLVRIAIQNLLYYGVVTLVSILQYSNVYCPTPKVQDLVDDKSLQEACLSYVTKQGHKRASLRDVFQLYCSLSPGTTVRDLIGRHPQQLQHVDERKLIQFGLMKNLIRRLQKYPVRVTREEQSHPARLYTGCHSYDEICCKTGMSYHELDERLENDPNIIICWK